(19) United States
(12) Patent Application Publication
(10) Pub. No.: US 2025/0235437 A1
(43) Pub. Date: Jul. 24, 2025

(54) PROTEOLYSIS TARGETING CHIMERA (PROTAC) MOLECULE FOR DEGRADATION OF ENL AND CANCER THERAPY

(71) Applicant: Baylor College of Medicine, Houston, TX (US)

(72) Inventors: Yongcheng Song, Houston, TX (US); Xin Li, Houston, TX (US); Yuan Yao, Houston, TX (US)

(73) Assignee: Baylor College of Medicine, Houston, TX (US)

(21) Appl. No.: 18/849,494

(22) PCT Filed: Mar. 22, 2023
(86) PCT No.: PCT/US2023/015941

§ 371 Date: Sep. 21, 2024

Related U.S. Application Data
(60) Provisional application No. 63/323,001, filed on Mar. 23, 2022.

(51) Int. Cl. A61K 31/4439; A61P 35/02; C07D 401/14; G01N 33/50

(52) U.S. Cl. CPC A61K 31/4439; A61P 35/02; C07D 401/14; G01N 33/5011

(57) ABSTRACT

Embodiments of the present disclosure pertain to compounds that include: a molecule capable of binding to an ENL protein; and a ligand of an E3 ubiquitin ligase, where the molecule and the ligand are coupled to one another by a linker, a chemical bond, or combinations thereof. Additional embodiments of the present disclosure pertain to methods of treating or preventing a condition in a subject by administering to the subject a compound of the present disclosure. The condition to be treated or prevented may be associated with an ENL protein abnormality or facilitated by an ENL protein. Additional embodiments of the present disclosure pertain to methods of evaluating cellular activity by exposing a cell to a compound of the present disclosure.

### CROSS-REFERENCE TO RELATED APPLICATIONS

This application claims priority to U.S. Provisional Patent Application No. 63/323,001, filed on Mar. 23, 2022. The entirety of the aforementioned application is incorporated herein by reference.

### STATEMENT REGARDING FEDERALLY SPONSORED RESEARCH

This invention was made with government support under R01CA266057, awarded by the National Institutes of Health. The government has certain rights in the invention.

### STATEMENT UNDER 37 C.F.R. § 1.834(C)(1)

Pursuant to 37 C.F.R. § 1.834, Applicant hereby submits a sequence listing as an XML file (“Sequence Listing”). The name of the file containing the Sequence Listing is “AF44111.P037WO.xml”. The date of the creation of the Sequence Listing is Mar. 22, 2023. The size of the Sequence Listing is 3,000 bytes. Applicant hereby incorporates by reference the material in the Sequence Listing.

### BACKGROUND

Current methods and therapeutics for treating, preventing or detecting conditions associated with ENL proteins or their abnormalities suffer from numerous limitations. Embodiments of the present disclosure aim to address the aforementioned limitations.

### SUMMARY

In some embodiments, the present disclosure pertains to a compound that includes: a molecule capable of binding to an ENL protein; and a ligand of an E3 ubiquitin ligase, where the molecule and the ligand are coupled to one another by a linker, a chemical bond, or combinations thereof. In some embodiments, the compounds of the present disclosure may be suitable for use in treating or preventing a condition in a subject. In some embodiments, the condition is associated with an ENL protein abnormality or facilitated by an ENL protein.

Additional embodiments of the present disclosure pertain to methods of treating or preventing a condition in a subject by administering to the subject a compound of the present disclosure. In some embodiments, the condition to be treated or prevented is associated with an ENL protein abnormality or facilitated by an ENL protein. In some embodiments, the condition to be treated or prevented is cancer. In some embodiments, the cancer includes, without limitation, a cancer facilitated by an ENL protein, a cancer associated with an ENL protein abnormality, leukemia, acute lymphocytic leukemia (ALL), myeloid leukemia (AML), mixed lineage leukemia 1 (MLL1), MLL1-rearranged (MLL1-r) ALL, Wilms tumor, kidney cancer, or combinations thereof. In some embodiments, the cancer includes mixed lineage leukemia 1 (MLL1).

Additional embodiments of the present disclosure pertain to methods of evaluating cellular activity by exposing a cell to a compound of the present disclosure. In some embodiments, the cells include cancer cells. In some embodiments, the method is utilized to evaluate the ability of the compounds of the present disclosure to interfere with the carcinogenesis.

### DESCRIPTION OF THE DRAWINGS

FIG. 1 provides an illustration of ENL and its paralog AF9 with associated proteins. The representative inhibitors of these protein-protein interactions are also shown.

FIG. 2 provides structures and synthesis of compounds 1-4 with reagents and conditions: (i) Ethyl chloroacetate, 4N HCl (aq.), 100° C., 94%; (ii) (2S)-2-Methyl-pyrrolidine, Na₂CO₃, CH₃CN, 70%; (iii) H₂, 10% Pd/C, MeOH; (iv) NaH, N-(6-bromohexyl) phthalimide, dimethylformamide, 47.7%; (v) 37% HCl (aq.), 100° C.; (vi) 1-[Bis(dimethylamino)methylene]-1H-1,2,3-triazolo[4,5-b]pyridinium 3-oxide hexafluorophosphate, diisopropylethylamine, dichloromethane, 25° C., 65%; (vii) NH₂NH₂, EtOH, 50° C., 89%; (viii) 2-(2,6-dioxo-3-piperidinyl)-4-fluoro-1H-isoindole-1,3(2H)-dione, diisopropylethylamine, dimethyl sulfoxide, 90° C., 44%; and (ix) 1-adamantaneacetic acid, condition vi, 64.4%.

FIGS. 3A-3I show the activity of compounds 1-3 on ENL and AF9 in MLL1-r leukemia cells. FIGS. 3A-3E show the levels of ENL, AF9 and β-actin (as a control) in MV4;11 cells (FIGS. 3A-3D) and Molm-13 cells (FIG. 3E) upon treatment with compounds 1 (FIGS. 3A, 3D, and 3E), 2 (FIG. 3B), and 3 (FIG. 3C) at the specified concentrations for 24 h, showing they induced degradation of ENL with their dose-responsive curves for calculating DC₅₀ shown at right. AF9 levels were not reduced. FIG. 3F shows time-dependent degradation of ENL in MV4;11 cells by compound 1 (500 nM). FIGS. 3G-3I show ENL levels in MV4;11 cells upon pre-treatment for 2 h with SGC-iMLLT (Inh, FIG. 3G), thalidomide (Tha, FIG. 3H) and bortezomib (Bor, FIG. 3I) followed by co-treatment with compound 1 (500 nM) for 24 h, showing these three compounds can dose-dependently inhibit compound 1-mediated ENL degradation.

FIGS. 4A-4G show that compound 1 reduced ENL, but not other SEC proteins in MLL1-r leukemia cells. FIGS. 4A-4B show that, upon compound treatment at the specified concentrations for 4 days, levels of ENL and other related proteins in the nucleus (FIG. 4A) and cytoplasm (FIG. 4B) of MV4;11 cells, showing compound 1 only significantly reduced ENL. FIGS. 4C-4G show ChIP-qPCR results showing the enrichment of ENL (FIG. 4C), AF9 (FIG. 4D), AFF4 (FIG. 4E), cyclin-Tl (FIG. 4F) and H3K79me2 (FIG. 4G) in the gene promoters of Myc and HoxA9. Treatment with compound 1 (500 nM) only significantly reduced the binding of ENL to these gene promoters. (*p<0.05).

FIGS. 5A-5D show that compound 1 inhibited malignant gene expression in MLL1-r leukemia Molm-13 cells. FIGS. 5A-5C show that treatment with compound 1 for 4 days dose-dependently inhibited expression of HoxA9 (FIG. 5A), Meis1 (FIG. 5B), and Myc (*p<0.05) (FIG. 5C). FIG. 5D shows that gene profiling followed by gene set enrichment analysis (GSEA) demonstrate that treatment of Molm-13 cells with compound 1 (500 nM for 4 days) recapitulated activities of ENL knockdown (GSE80774, panels 1 and 2) and knockdown of MLL1-AF9 and MLL1-ENL (GSE36592, panels 3 and 4). Compound 1 also significantly reversed expression of HoxA9-regulated target genes (GSE13714, panels 5 and 6), and downregulated Myc target genes (GSE32220, panel 7).

FIGS. 6A-6G summarize the antitumor activities of ENL-targeting compounds. FIG. 6A summarizes the antiproliferative activities of the compounds upon 7-day incubation, showing compound 1 inhibited proliferation of MLL1-r leukemia (Molm-13 and MV4;11), Myc-driven Kasumi-1 (AML), RPMI8226, and U266 (myeloma) cells, while it had no activity against solid Hela (cervical) and Panc1 (pancreatic) cancer cells. FIG. 6B shows time-dependent activity of compound 1 against proliferation of Molm-13 cells. FIGS. 6C-6D shows treatment of Molm-13 cells with compound 1 led to dose-dependent apoptosis (FIG. 6C) and differentiation (at 3 μM) (FIG. 6D) with more cells expressing high levels of CD14 (upper) and CD11b (lower). FIG. 6E shows that treatment with compound 1 (30 mg/kg/day for 13 days) caused no significant changes in blood cell counts. FIGS. 6F-6G show that treatment with compound 1 (30 mg/kg/day for Day-3-15) significantly inhibited tumor growth (FIG. 6F) with prolonged survivals (FIG. 6G) in mice with subcutaneously xenografted Molm-13 leukemia.

FIGS. 7A-7D show that compound 1 degraded mutant ENL (mENL) and suppressed its mediated gene transcription. FIG. 7A shows cellular levels of mutant/wild-type (WT) ENL and R-actin detected by (left panel) FLAG or (right panel) ENL antibody, upon transfection with increasing amounts of a mENL-containing plasmid for 4 h followed by 24 h incubation, showing dose-dependent expression of mENL. Endogenous WT ENL can also be detected and included in quantification (right panel). FIGS. 7B-7C show levels of mutant/WT ENL detected by a FLAG or ENL antibody, upon transfection with 0.1 (FIG. 7B) or 0.2 μg (FIG. 7C) of the plasmid for 4 h followed by 24 h treatment with compound 1, showing the compound can dose-dependently degrade or deplete both mutant and WT ENL. FIG. 7D shows transfection with 0.04 g of the plasmid for 4 h followed by 24 h incubation upregulated expression of HoxA11 (left) and HoxA13 (right), and treatment with 1 during the incubation inhibited such gene overexpression (*p<0.05).

FIG. 8 provides exemplary structures of the compounds of the present disclosure.

FIGS. 9A-9E illustrate the activities of compounds SYC-2552 (FIG. 9A), SYC-2553 (FIG. 9B), SYC-2555 (FIG. 9C), SYC-2556 (FIG. 9D), and SYC-2557 (FIG. 9E) in degrading ENL or AF9 in MV4;11 cells.

FIGS. 10A-10H illustrate the activities of compounds SYC-2552 (FIG. 10A), SYC-2553 (FIG. 10B), SYC-2554 (FIG. 10C), SYC-2555 (FIG. 10D), SYC-2556 (FIG. 10E), SYC-2557 (FIG. 10F), and SYC-2558 (FIGS. 10G-10H) in degrading ENL or AF9 in Molm13 cells.

FIGS. 11A-11B show that compound SYC-2229 inhibits malignant gene expression in RPMI-8226 cells. FIG. 11A shows treatment with SYC-2229 for 4 days dose-dependently inhibited expression of Myc (*p<0.05). FIG. 11B shows that gene profiling followed by gene set enrichment analysis (GSEA) demonstrates that treatment of RPMI-8226 cells with SYC-2229 (500 nM for 4 days) recapitulated activities of ENL knockdown (GSE80774, panels 1 and 2) and downregulated Myc target genes (GSE80774, panels 3 and 4).

### DETAILED DESCRIPTION

It is to be understood that both the foregoing general description and the following detailed description are illustrative and explanatory, and are not restrictive of the subject matter, as claimed. In this application, the use of the singular includes the plural, the word “a” or “an” means “at least one”, and the use of “or” means “and/or”, unless specifically stated otherwise. Furthermore, the use of the term “including”, as well as other forms, such as “includes” and “included”, is not limiting. Also, terms such as “element” or “component” encompass both elements or components comprising one unit and elements or components that include more than one unit unless specifically stated otherwise.

The section headings used herein are for organizational purposes and are not to be construed as limiting the subject matter described. All documents, or portions of documents, cited in this application, including, but not limited to, patents, patent applications, articles, books, and treatises, are hereby expressly incorporated herein by reference in their entirety for any purpose. In the event that one or more of the incorporated literature and similar materials defines a term in a manner that contradicts the definition of that term in this application, this application controls.

Numerous conditions are associated with ENL protein activities or abnormalities. For instance, chromosome translocations involving mixed lineage leukemia 1 (MLL1, also known as MLL or KMT2A) cause acute leukemia in infants, children and adults with adverse clinical outcomes. In particular, acute lymphocytic leukemia (ALL) and myeloid leukemia (AML) caused by chromosome translocations of MLL1 account for about 70% of the diseases in infants and 5-10% in children and adults with a poor prognosis. Five-year survival rates for MLL1-rearranged (MLL1-r) ALL are about 35%, as compared with about 90% for other pediatric ALLs. MLL1-r AML also carries poor clinical outcomes with five-year survivals of about 30%. Although a significant progress has been achieved to understand the biology of MLL1-r leukemias, more effective treatments are needed.

Despite the identification of over 70 fusion partners of MLL1, only a few are frequently found in about 70% MLL1-r leukemias, including transcription cofactors AF9 (also known as MLLT3) and its paralog ENL (also known as MLLT1), AF4 and its paralog AFF4, and ELL. Together with the cyclin-T1/CDK9 complex (also known as P-TEFb), these proteins associate with each other and constitute super elongation complexes (SEC), which promote malignant gene expression (e.g., HoxA9, Meis1 and Myc) in MLL1-r leukemia and play critical roles in the cancer initiation and maintenance.

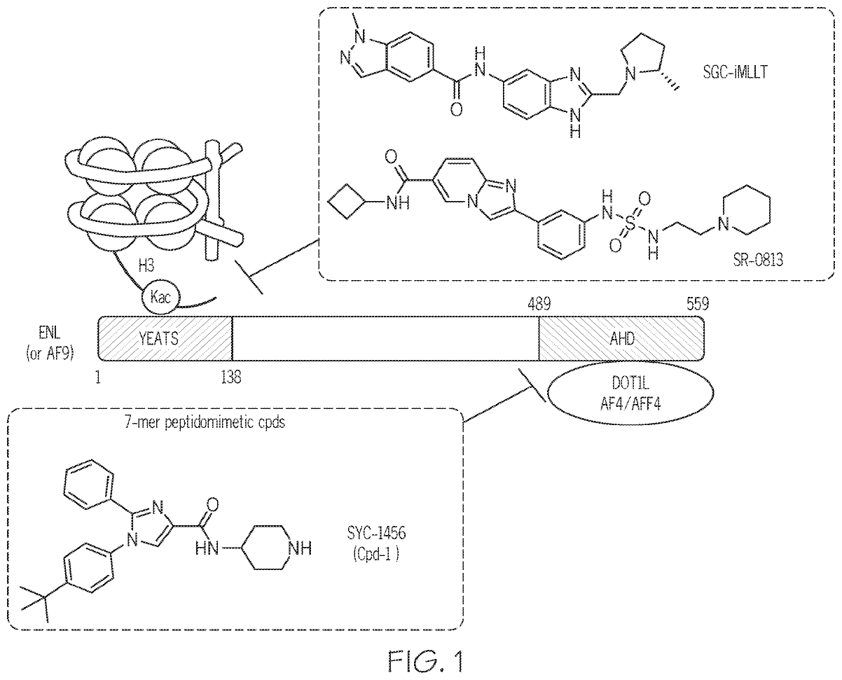

ENL and homologous AF9 contain an N-terminal YEATS, a central intrinsically disordered linker and C-terminal AHD domain (FIG. 1). The YEATS domain recognizes an acetylated histone lysine residue (e.g., H3K27ac) and such binding has been found to be important in gene regulation. The less conserved, long linker regions of ENL and AF9 have been poorly studied. While YEATS is lost in most clinical variances of MLL1-AF9/-ENL and dispensable for the leukemia, the AHD domain is always present in the fusion oncogenes and required for leukemogenesis. Recognizing a consensus sequence of LxVxIxLxxV/L, ENL/AF9 AHD can bind AF4/AFF4 or histone H3K79 methyltransferase DOT1L with a high affinity. Thus, in addition to forming SEC for transcription elongation, AHD can recruit DOT1L for hypermethylation of H3K79, which is characteristic and critical to MLL1-r leukemia. Moreover, ENL/AF9 AHD can also bind CBX8 (chromobox homolog 8) or BCoR (BCL-6 corepressor) and such protein-protein interactions have been reported to be important for MLL1-AF9/-ENL mediated leukemogenesis. Interestingly, despite their high homology (particularly in the YEATS and AHD domains), ENL functions differently from AF9 with knockout studies showing that ENL, but not AF9, is critical to MLL1-r leukemia and other AMLs.

Recently, recurrent mutations in the YEATS domain of ENL have been found in Wilms tumor, the most common pediatric kidney cancer. Dysregulated expression of certain Hox genes and Myc is characteristic to ENL-mutated Wilms tumors. Further studies show the mutation induced self-association of the mutant ENL. Moreover, there was significantly increased binding of the mutant ENL-associated SEC to these gene loci, causing aberrant gene transcription and eventually oncogenesis.

Much interest has been generated to pharmacologically inhibit ENL/AF9. Several potent small-molecule inhibitors of YEATS were reported to disrupt the ENL/AF9-H3K27ac interaction. Several 7-mer peptidomimetic compounds and a small molecule compound SYC-1456 are inhibitors of the AHD domain. SYC-1456 can suppress onco-MLL1 mediated aberrant gene expression, induce cell differentiation and apoptosis, and inhibit tumor growth in cell and mouse models of MLL1-r leukemia, thereby validating that ENL inhibition is a viable therapeutic approach.

However, there remains limited therapeutic drugs that specifically target ENL proteins in order to detect, treat or prevent ENL-related conditions. Numerous embodiments of the present disclosure address the aforementioned limitation.

### Compounds

In some embodiments, the present disclosure pertains to compounds. In some embodiments, the compounds of the present disclosure are suitable for use in treating or preventing a condition in a subject. In some embodiments, the condition is associated with an ENL protein abnormality or facilitated by an ENL protein.

In some embodiments, the compounds of the present disclosure generally include: (1) a molecule capable of binding to an ENL protein (“molecule”); and (2) a ligand of an E3 ubiquitin ligase (“ligand”). In some embodiments, the molecule and the ligand are coupled to one another by a linker, a chemical bond, or combinations thereof.

The molecules of the present disclosure can bind to various types of ENL proteins. For instance, in some embodiments, the ENL protein includes SEQ ID NO: 1, a sequence that shows at least 65% similarity to SEQ ID NO: 1, a derivative thereof, a homologue thereof, an analogue thereof, or combinations thereof. In some embodiments, the ENL protein includes a sequence that shows at least 70% similarity to SEQ ID NO: 1. In some embodiments, the ENL protein includes a sequence that shows at least 75% similarity to SEQ ID NO: 1. In some embodiments, the ENL protein includes a sequence that shows at least 80% similarity to SEQ ID NO: 1. In some embodiments, the ENL protein includes a sequence that shows at least 85% similarity to SEQ ID NO: 1. In some embodiments, the ENL protein includes a sequence that shows at least 90% similarity to SEQ ID NO: 1. In some embodiments, the ENL protein includes a sequence that shows at least 95% similarity to SEQ ID NO: 1.

The molecules of the present disclosure can include numerous structures. For instance, in some embodiments, the molecules of the present disclosure include a structure of:

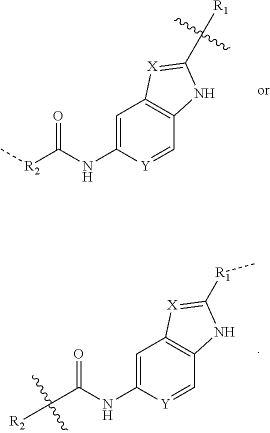

In some embodiments, each of X and Y in the aforementioned structure independently includes N or CH. In some embodiments, X is N and Y is CH. In some embodiments, X and Y are CH. In some embodiments, X and Y are N.

In some embodiments, R₁ in the aforementioned structures includes a functional group. In some embodiments, the functional group includes, without limitation:

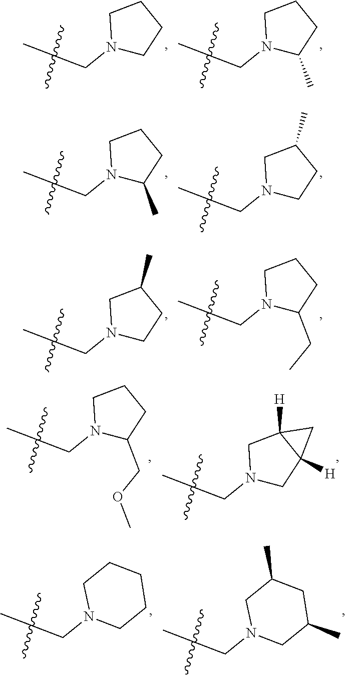

derivatives thereof, or combinations thereof.

In some embodiments, R2 in the aforementioned structures also includes a functional group. In some embodiments, the functional group includes, without limitation:

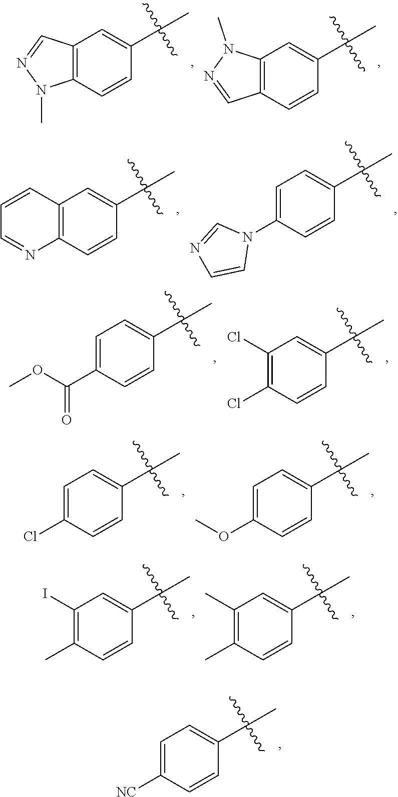

derivatives thereof, or combinations thereof.

In some embodiments, [symbol] in the aforementioned structures represents a chemical bond or linker that couples the molecule to the linker. In some embodiments, [symbol] represents a chemical bond between the molecule and the ligand. In some embodiments, [symbol] represents a linker.

In some embodiments, the linker includes a structure that includes, without limitation:

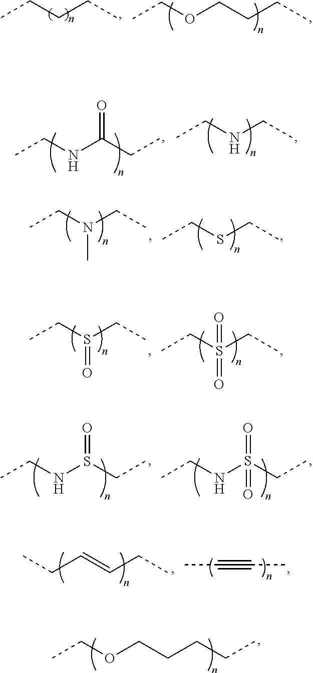

derivatives thereof, or combinations thereof.

In some embodiments, n is an integer of 1 or greater. In some embodiments, n is an integer between 1-20. In some embodiments, the linkers of the present disclosure have one or more of the following structures:

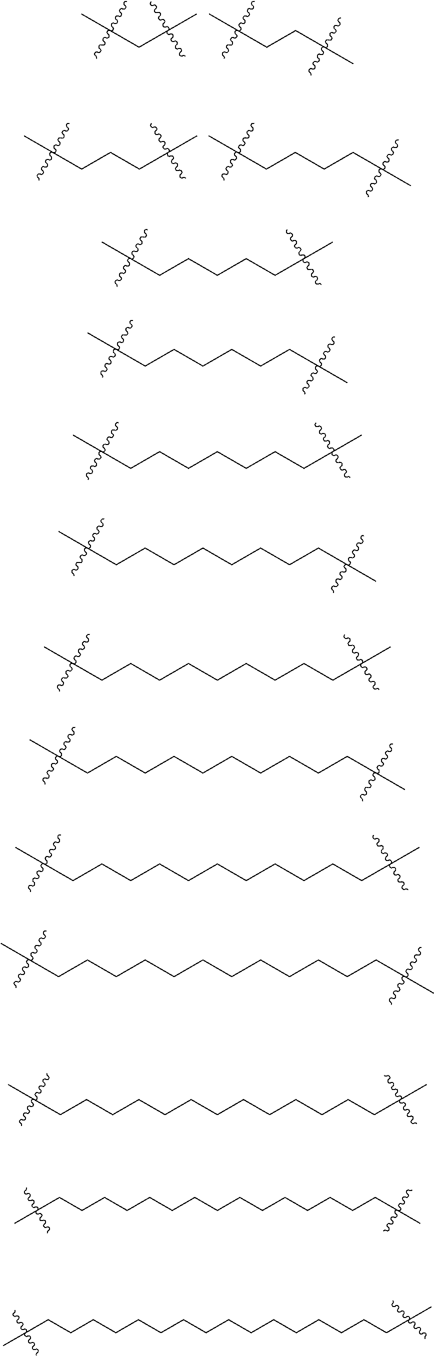

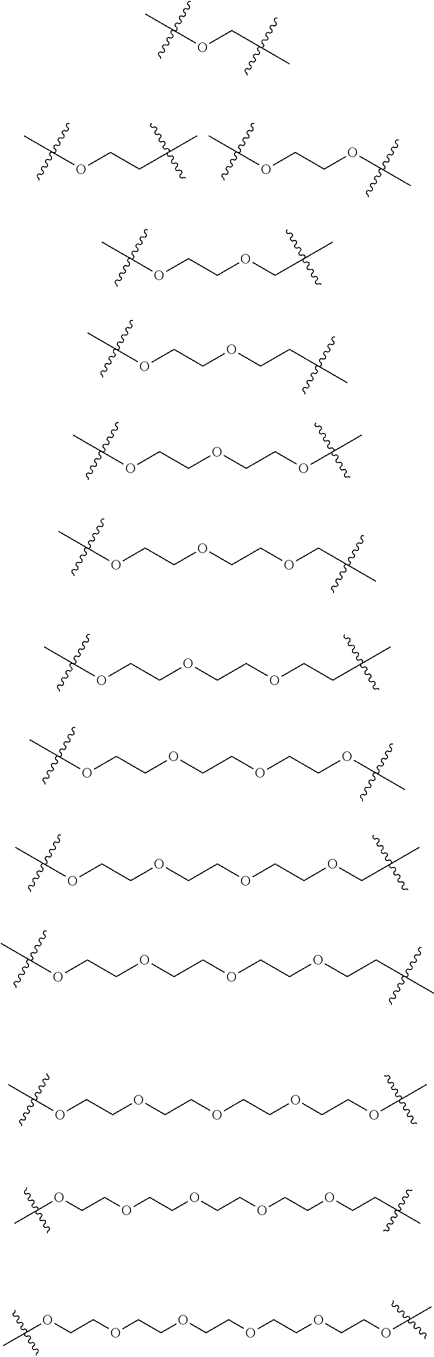

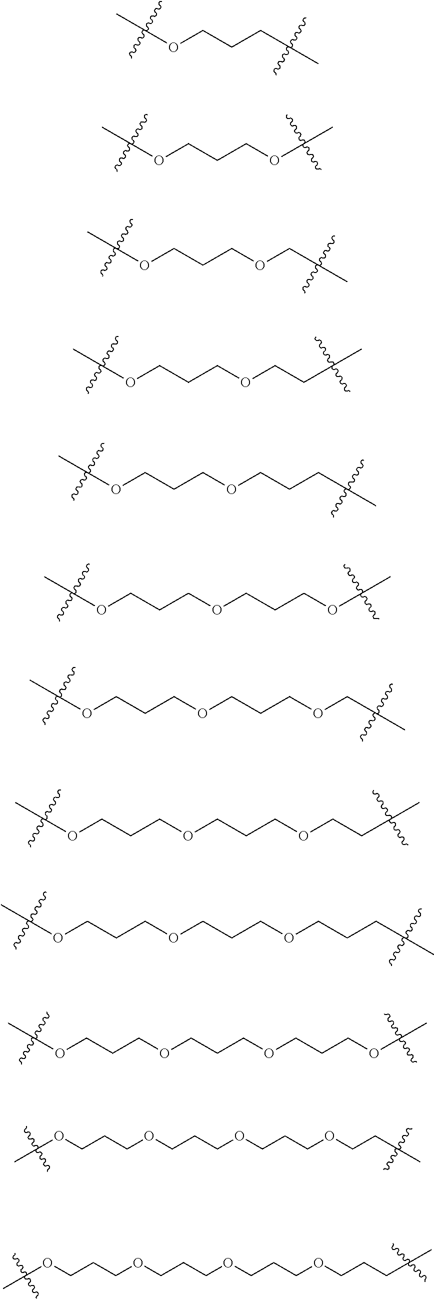

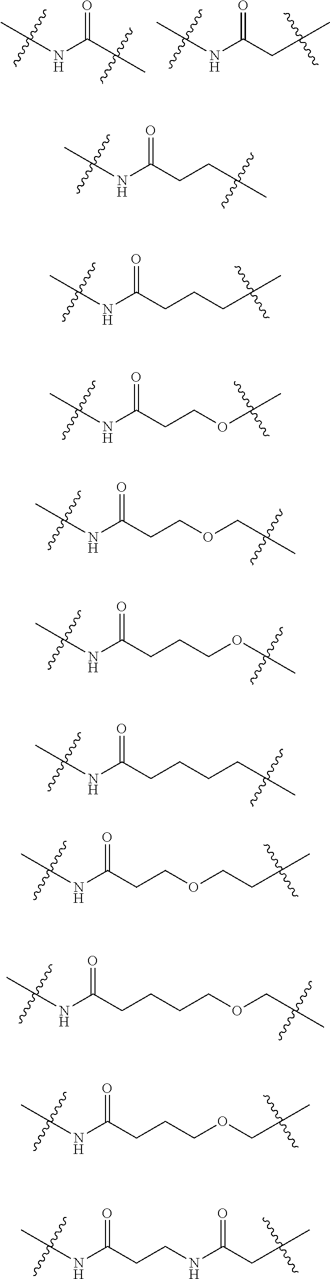

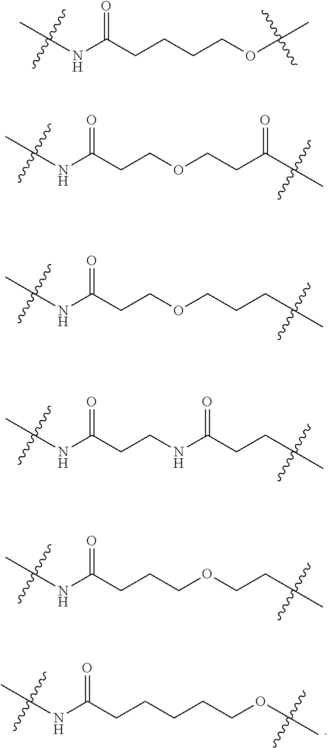 [SMILES: *NC(=O)CCCCO* ; *NC(=O)CCOCCC(*)=O ; *CCCOCCC(=O)N* ; *CCC(=O)NCCC(=O)N* ; *CCOCCCC(=O)N* ; *NC(=O)CCCCCO*]

The compounds of the present disclosure can also include various types of ligands. For instance, in some embodiments, the ligand includes, without limitation, a von Hippel-Lindau disease tumor suppressor protein (VHL) ligand, a cereblon (CRBN) ligand, the mouse double minute 2 homologue (MDM2), inhibitor of apoptosis (IAP) ligand, or combinations thereof.

In some embodiments, the ligand includes a VHL ligand. In some embodiments, the VHL ligand includes, without limitation:

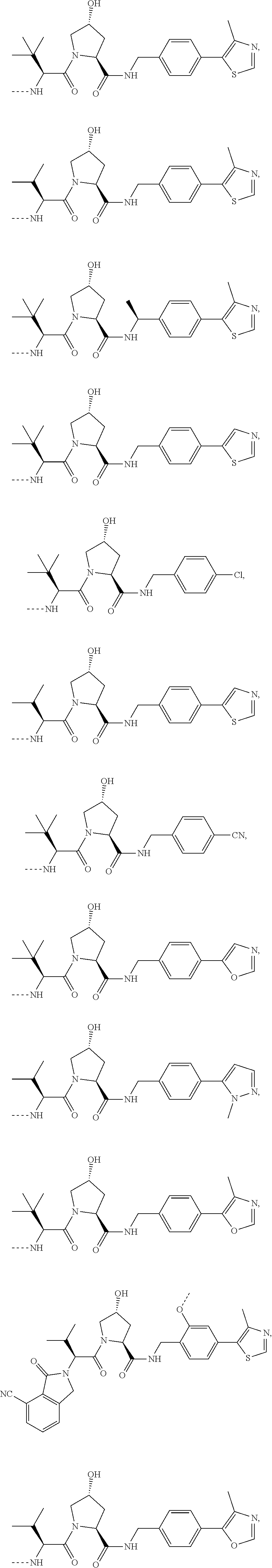

derivatives thereof, or combinations thereof.

In some embodiments, [symbol] represents a chemical bond or a linker. Suitable chemical bonds and linkers were described previously in this Application.

In some embodiments, the ligand includes a CRBN ligand. In some embodiments, the CRBN ligand includes, without limitation:

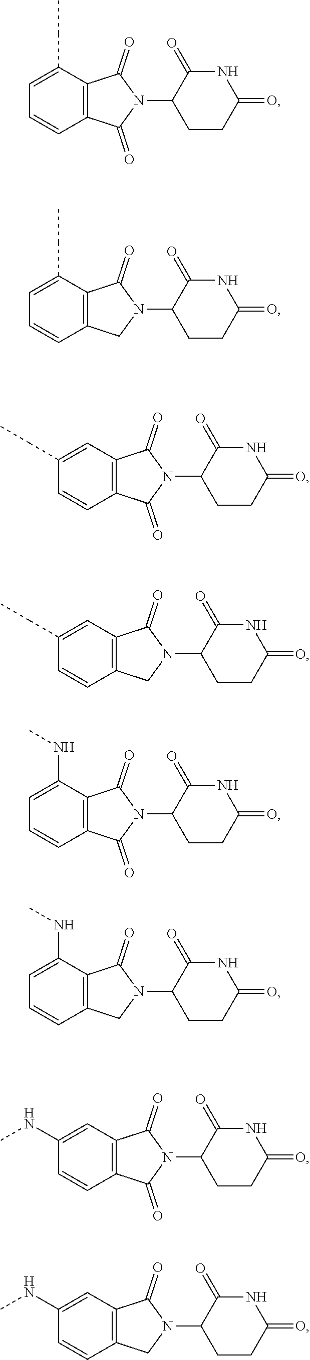

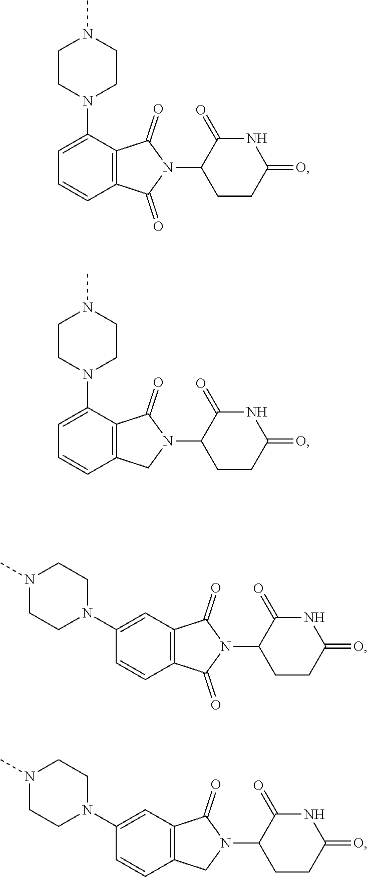

derivatives thereof, or combinations thereof.

In some embodiments, [symbol] represents a chemical bond or a linker. Suitable chemical bonds and linkers were described previously in this Application.

In some embodiments, the compounds of the present disclosure include the following structure:

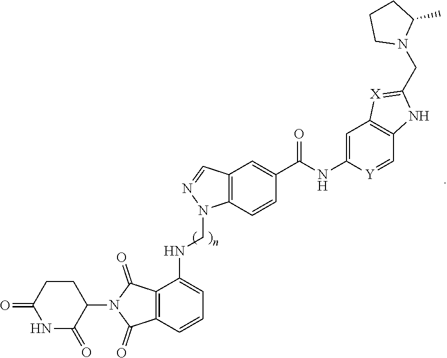

In some embodiments, each of X and Y in the aforementioned structure independently includes N or CH. In some embodiments, X is N and Y is CH. In some embodiments, X is CH and Y is N. In some embodiments, X and Y are CH. In some embodiments, X and Y are N.

In some embodiments, n is an integer of 0 or greater. In some embodiments, n is 3, 4, 5, 6, 7, 8, 9, 10 or 11. In some embodiments, n is 4, 6, or 8. In some embodiments, n is 8. Exemplary compounds of the present disclosure are also illustrated in FIG. 8.

In some embodiments, the compounds of the present disclosure are in a composition. In some embodiments, the compounds of the present disclosure are in a composition at a concentration sufficient to treat or prevent a condition in a subject, where the condition is associated with an ENL protein abnormality or facilitated by an ENL protein. In some embodiments, the compounds of the present disclosure are at a concentration of at least about 5 wt % in the composition. In some embodiments, the compounds of the present disclosure are at a concentration of at least about 10 wt % in the composition. In some embodiments, the compounds of the present disclosure are at a concentration of at least about 15 wt % in the composition. In some embodiments, the compounds of the present disclosure are at a concentration of at least about 20 wt % in the composition. In some embodiments, the compounds of the present disclosure are at a concentration of at least about 25 wt % in the composition.

The compositions of the present disclosure can include various constituents. For instance, in some embodiments, the compositions of the present disclosure also include an active agent stabilizer. In some embodiments, the active stabilizer can include, without limitation, an anti-oxidant. In some embodiments, the anti-oxidant includes, without limitation, vitamin E, vitamin C, vitamin A, triglyceride, uric acid, glutathione, and combinations thereof.

In some embodiments, the compositions of the present disclosure can also include excipients. In some embodiments, the excipients include, without limitation, triglycerides, monosaccharides, disaccharides, polysaccharides, fibers, lipids, vitamins, minerals, phytochemicals, proteins, terpenoids, and combinations thereof. In some embodiments, the compositions of the present disclosure are in the form of a pill.

### Treatment or Prevention of Conditions

Additional embodiments of the present disclosure pertain to methods of treating or preventing a condition in a subject by administering to the subject one or more compounds of the present disclosure. The methods of the present disclosure can be utilized to treat or prevent various conditions. For instance, in some embodiments, the condition is associated with an ENL protein abnormality or facilitated by an ENL protein. In some embodiments, the ENL protein includes SEQ ID NO: 1, a sequence that shows at least 65% similarity to SEQ ID NO: 1, a derivative thereof, a homologue thereof, an analogue thereof, or combinations thereof.

In some embodiments, the condition to be treated or prevented is associated with an ENL protein abnormality. In some embodiments, the ENL protein abnormality is characterized by overexpression of the ENL protein, under-expression of the ENL protein, mutation of the ENL protein, or combinations thereof.

In some embodiments, the condition to be treated or prevented is facilitated by an ENL protein. In some embodiments, the ENL protein facilitates, propagates, or causes the condition.

In some embodiments, the condition to be treated or prevent is a cancer. In some embodiments, the cancer includes, without limitation, a cancer facilitated by an ENL protein, a cancer associated with an ENL protein abnormality, leukemia, acute lymphocytic leukemia (ALL), myeloid leukemia (AML), mixed lineage leukemia 1 (MLL1), MLL1-rearranged (MLL1-r) ALL, Wilms tumor, kidney cancer, or combinations thereof. In some embodiments, the cancer includes mixed lineage leukemia 1 (MLL1).

In some embodiments, the methods of the present disclosure can be used to treat a certain condition. In some embodiments, the methods of the present disclosure can be used to prevent a certain condition. In some embodiments, the methods of the present disclosure can be used to treat and prevent a certain condition.

The methods of the present disclosure can be utilized to treat and/or prevent conditions in various subjects. For instance, in some embodiments, the subject is a human being. In some embodiments, the subject is suffering from the condition. In some embodiments, the subject is vulnerable to the condition.

In some embodiments, the methods of the present disclosure also include a step of instructing the subject to administer the compounds of the present disclosure in order to treat or prevent the condition in the subject. In some embodiments, the instructing occurs by providing the subject with written instructions. In some embodiments, the instructing occurs by providing the subject with oral instructions.

Various methods may be utilized to administer the compounds of the present disclosure to a subject. For instance, in some embodiments, the administering occurs by a method that includes, without limitation, intravenous administration, intramuscular administration, intradermal administration, intraperitoneal administration, subcutaneous administration, spray-based administration, aerosol-based administration, in ovo administration, oral administration, intraocular administration, intratracheal administration, intranasal administration, inhalational administration, local administration, and combinations thereof. In some embodiments, the administering occurs by oral administration.

In some embodiments, the administering occurs by local administration to a certain region of a subject affected by a condition. For instance, in some embodiments, the administering occurs by local administration to a site of a tumor in a subject.

### Mechanisms of Action

Without being bound by theory, the compounds of the present disclosure can treat or prevent conditions in a subject through various mechanisms of action. For instance, in some embodiments, the compounds of the present disclosure act as a proteolysis targeting chimeric molecule (PROTAC) compound, where the molecule of the compound binds to an ENL protein, and the ligand of the compound binds to an E3 ligase. Thereafter, the E3 ligase catalyzes the transfer of ubiquitin to the ENL protein. This in turn results in the degradation of the ENL protein by the ubiquitin-proteasome pathway.

### Methods of Evaluating Cellular Activity

Additional embodiments of the present disclosure include methods of evaluating cellular activity by exposing a cell to one or more compounds of the present disclosure. The methods of the present disclosure can be utilized to evaluate various types of cellular activities.

In some embodiments, the exposing of the compounds of the present disclosure to cells occurs in vitro. As such, in some embodiments, the methods of the present disclosure may be utilized to evaluate a cellular activity in vitro.

In some embodiments, the exposing of the compounds of the present disclosure to cells occurs in vivo in a subject. As such, in some embodiments, the methods of the present disclosure may be utilized to evaluate a cellular activity in vivo.

In some embodiments, the cells that are exposed to the compounds of the present disclosure include cancer cells. As such, in some embodiments, the cellular activity to be evaluated includes carcinogenesis. In some embodiments, the methods of the present disclosure may be utilized to evaluate the ability of the compounds of the present disclosure to interfere with the carcinogenesis.

In some embodiments, the cells that are exposed to the compounds of the present disclosure include normal cells. As such, in some embodiments, the cellular activity to be evaluated includes toxicity or development. In some embodiments, the methods of the present disclosure may be utilized to evaluate the ability of the compounds of the present disclosure to cause toxicities or to interfere with the development.

### ADDITIONAL EMBODIMENTS

Reference will now be made to more specific embodiments of the present disclosure and experimental results that provide support for such embodiments. However, Applicant notes that the disclosure below is for illustrative purposes only and is not intended to limit the scope of the claimed subject matter in any way.

### Example 1. A Proteolysis-Targeting Chimera Molecule Selectively Degrades ENL and Inhibits Malignant Gene Expression and Tumor Growth

Proteolysis-targeting chimera (PROTAC) technology has recently attracted much interest in drug discovery. With good cell permeability, a PROTAC molecule may cause proteasome-mediated degradation of its target protein, which complements pharmacological inhibition with a distinct mechanism of action. It also has other potential benefits, such as sub-stoichiometric activity and more selectivity.

In this Example, Applicant reports a PROTAC molecule that can cause efficient and selective degradation of ENL (but not AF9), resulting in inhibition of malignant gene signatures and proliferation of MLL1-r leukemia in vitro and in vivo. The molecule tested efficiently degraded ENL with DC₅₀ of 37 nM and almost depleted it at ˜500 nM in blood and solid tumor cells. AF9 (as well as other proteins in SEC) was not significantly decreased. Compound-mediated ENL reduction significantly suppressed malignant gene signatures, selectively inhibited cell proliferation of MLL1-r leukemia and Myc-driven cancer cells with EC50s as low as 320 nM, and induced cell differentiation and apoptosis. The compounds exhibited significant antitumor activity in a mouse model of MLL1-r leukemia. The compounds can also degrade a mutant ENL in Wilms tumor and suppress its mediated gene transcription. As such, the compounds tests are novel chemical probes for cellular and in vivo studies of ENL (including its oncogenic mutants) and a lead compound for further anticancer drug development.

### Example 1.1. Compound Design and Synthesis

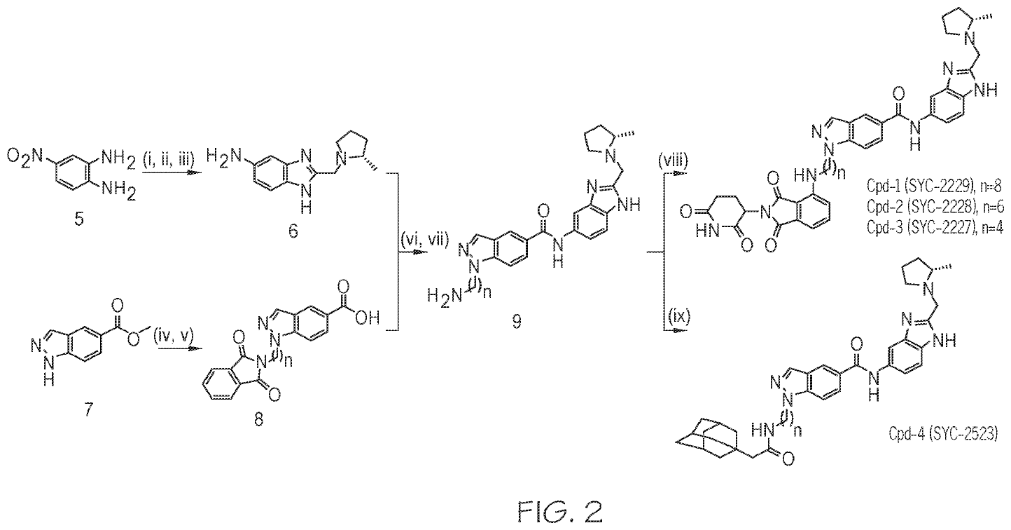

The designed PROTAC molecules consist of a YEATS inhibitor SGC-iMLLT and covalently linked thalidomide, a commonly used ligand of E3 ubiquitin ligase Cereblon. It is expected that, upon binding to ENL, the PROTAC compound can recruit Cereblon through its thalidomide moiety to form a ternary complex for ubiquitination of ENL, which is subjected to proteasome-mediated degradation. Based on the X-ray structure of ENL in complex with SGC-iMLLT, Applicant designed compounds 1-3 (SYC-2229, -2228, -2227, FIG. 2) with their linkers having no steric conflicts with ENL. In addition, a hydrophobic tagging compound 4 was designed as the second strategy to degrade ENL.

The synthetic scheme of compounds 1-4 is also shown in FIG. 2. 4-Nitrobenzene-1,2-diamine (5) was reacted with ethyl 2-chloroacetate to give 2-chloromethyl-5-nitrobenzimidazole, which was subjected to a substitution reaction with (S)-2-methylpyrrolidine followed by reduction of —NO₂ to give compound 6. Substitution reaction between methyl 1H-indazole-5-carbonate (7) and N-(6-bromohexyl)phthalimide followed by hydrolysis produced compound 8, which was coupled with 6 to give, upon deprotection, compound 9. A nucleophilic substitution reaction between the —NH₂ of 9 and the ortho-F-substituted thalidomide afforded the target PROTAC compounds 1-3. An amide-forming reaction between 9 and 1-adamantaneacetic acid produced compound 4.

### Example 1.2. ENL-Targeting PROTACs Bind to ENL/AF9 YEATS

Using an ALPHA (amplified luminescent proximity homogeneous assay) assay, compounds 1-3 were evaluated for their inhibition of the ENL YEATS-H3K27ac interaction. Compound 1 strongly inhibited such protein-protein interaction with an IC₅₀ of 170 nM (Table Si), while it is weaker than the parent inhibitor SGC-iMLLT (IC₅₀=32 nM in our assay). Similarly, compounds 2 and 3 are also strong inhibitors (IC₅₀=100 and 610 nM). These results indicate the linker-thalidomide moieties of compounds 1-3 only slightly reduce the binding affinity of SGC-iMMT to ENL YEATS. The decreased affinity might be due to the entropy costs associated with the flexibly linked thalidomide. In addition, consistent with previous studies, compounds 1-3 were found to bind to AF9 YEATS with comparable affinities using a similar ALPHA assay.

### Example 1.3. Cpd-1 Efficiently Degrades ENL, but not its Paralog AF9

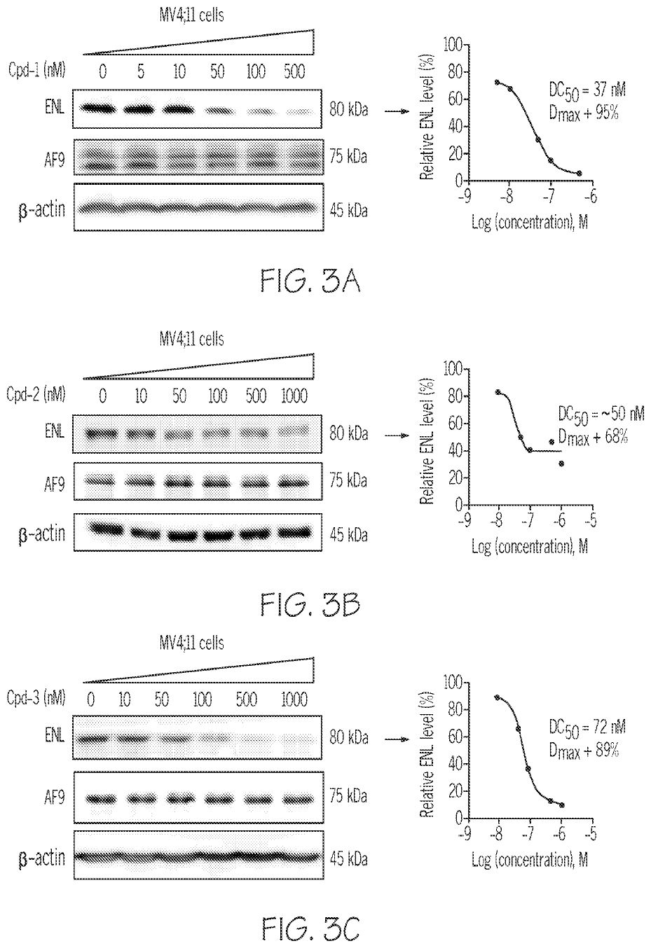

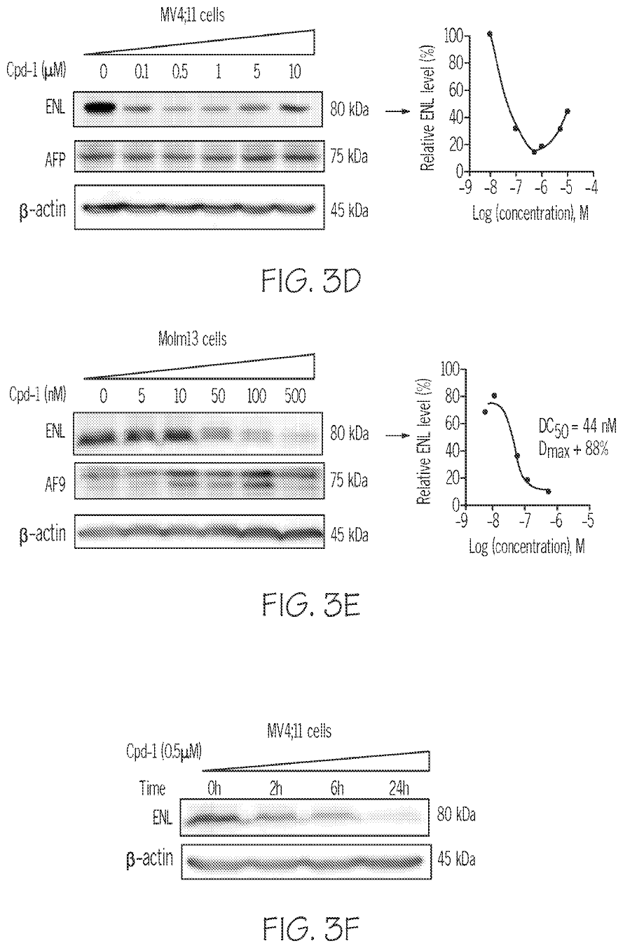

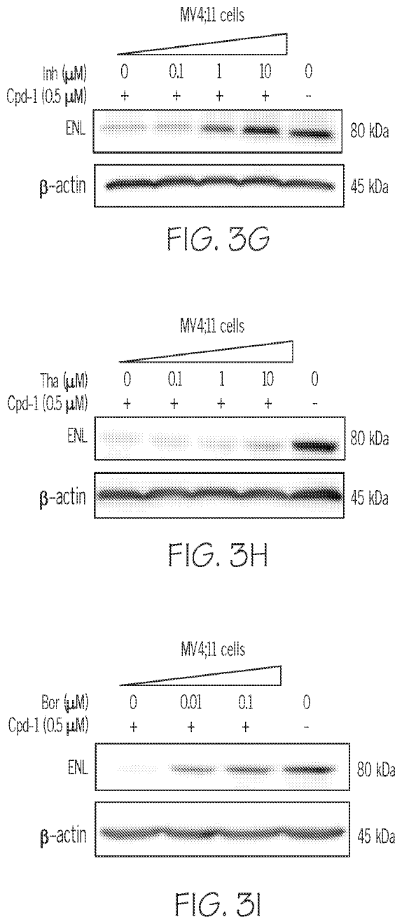

Next, Applicant tested whether the compounds degrade ENL and AF9 in MV4;11 leukemia cells with the .MLL1-AF4 oncogene. Upon compound treatment for 24 h, the cells were washed, lysed, and the lysates subjected to SDS-PAGE (sodium dodecyl sulphate-polyacrylamide gel electrophoresis) followed by detection with Western blot. As shown in FIGS. 3A and 3C, compounds 1 and 3 efficiently degraded ENL in a dose-dependent manner with DC₅₀ (concentration at which a protein is reduced by 50%) values of 37 and 72 nM, respectively, and almost depleted it at ˜500 nM with Dₘₐₓ (maximal degradation) of ˜95% and 89%. While compound 2 can degrade ENL with a DC₅₀ of ˜50 nM, it only reduced ENL by a maximum of ˜68% (FIG. 3B). Compound 4 did not reduce ENL even at 5 μM. Notably, AF9 levels in all these experiments were not reduced, showing compounds 1-3 are selective ENL-degrading probes.

The most active compound 1 was chosen for additional studies. It was found to exert the maximal activity at ˜500 nM, but the ENL level started to recover at higher concentrations from 1-10 μM (FIG. 3D).

This “hook” effect is commonly observed for a PROTAC because excessive compound 1 elevates inactive binary complexes ENL-1 and Cereblon-1, and decreases the active ternary complex ENL-1-Cereblon. In addition, compound 1 efficiently degraded ENL in another MLL1-r leukemia Molm-13 cells (with MLL1-AF9) with DC₅₀ of 47 nM and Dₘₐₓ of ˜88% (FIG. 3E). Moreover, significant ENL degradation can be detected in 2 h and depletion occurred within 24 h (FIG. 3F).

Mechanistically, SGC-iMLLT or thalidomide competitively inhibits the binding of 1 to ENL or Cereblon, respectively, while proteasome inhibitor bortezomib suppresses proteasome's activity to degrade ENL. All of these compounds should impair compound 1's ability to degrade ENL. MV4;11 cells were pre-treated with these three compounds for 2 h, followed by co-treatment with compound 1 (500 nM) for 24 h. As shown in FIGS. 3G-I, compound 1's ability to degrade ENL was compromised by the three compounds in a dose-dependent manner. These rescue experiments support compound 1 is a PROTAC-based ENL degrader.

### Example 1.4. Cpd-1 Only Reduces ENL, but not Other SEC Proteins in Cells or Gene Promoters

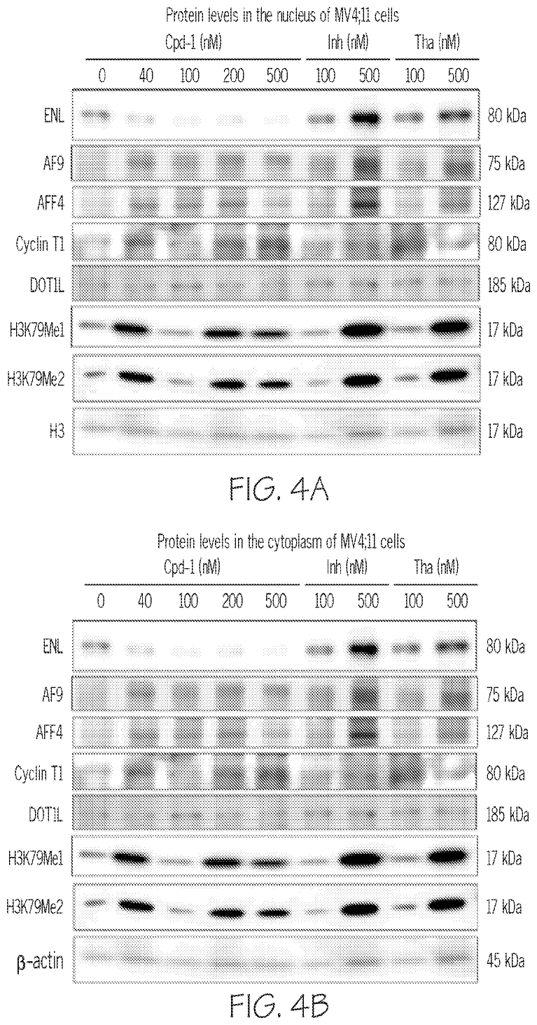

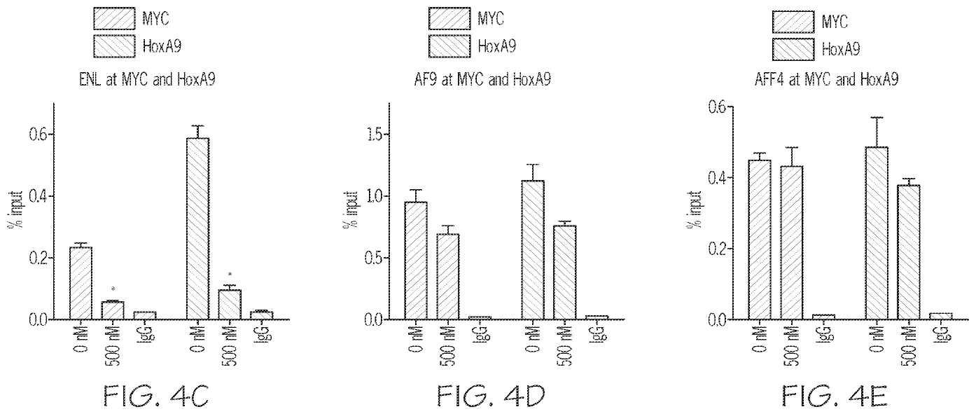

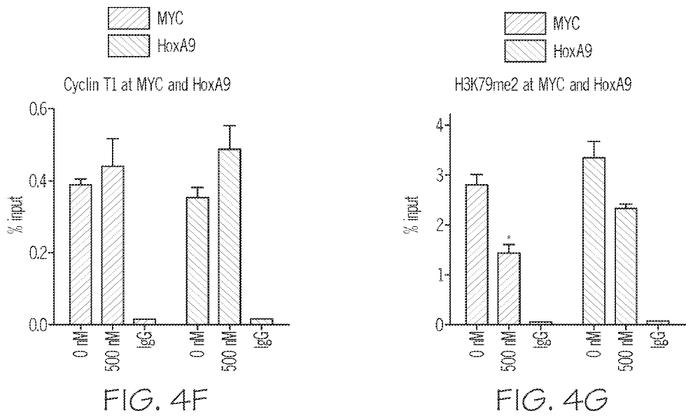

Applicant further investigated how compound 1 affects ENL, AF9 and other proteins of SEC in the cytoplasmic and nuclear compartments, the latter of which are more relevant to the functions of these transcription cofactors. SGC-iMLLT and thalidomide were included as controls for possible off-target effects. Upon treatment of MV4;11 cells with these compounds for 4 days, cytoplasmic and nuclear proteins were separated and subjected to SDS-PAGE/Western blot. As shown in FIG. 4A, ENL in the nucleus was significantly reduced dose-dependently and almost depleted with 500 nM of compound 1. SGC-iMLLT caused no reduction or even an increase of nuclear ENL, presumably because the inhibitor binds and helps stabilize ENL. Thalidomide did not reduce nuclear ENL. Moreover, AF9, AFF4 and cyclin-T1, three major components of SEC, as well as DOT1L (which binds AF9/ENL), were not significantly decreased by compound 1. In addition, nuclear levels of H3K79 methylation, the product of DOT1L catalyzed reactions, were not consistently affected by compound 1. The observed H3K79 methylation variations are puzzling but seem to be caused by off-target effects, as SGC-iMLLT or thalidomide caused similar changes. Similar protein changes were observed for the cytoplasmic extract, with only ENL levels were significantly reduced by compound 1 (FIG. 4B).

Chromatin immunoprecipitation (ChIP) followed by qPCR was used to further probe the activity of compound 1 in the gene promoters of Myc and HoxA9, two characteristic MLL1 target genes. As shown in FIGS. 4C-4G, compound 1 significantly reduced the ENL levels at these gene promoters, but in general it did not significantly lower down AF9, AFF4, cyclin-T1 and H3K79me2 at these gene loci.

### Example 1.5. Cpd-1-Mediated ENL Degradation Suppresses Malignant Gene Signatures in MLL1-r Leukemia

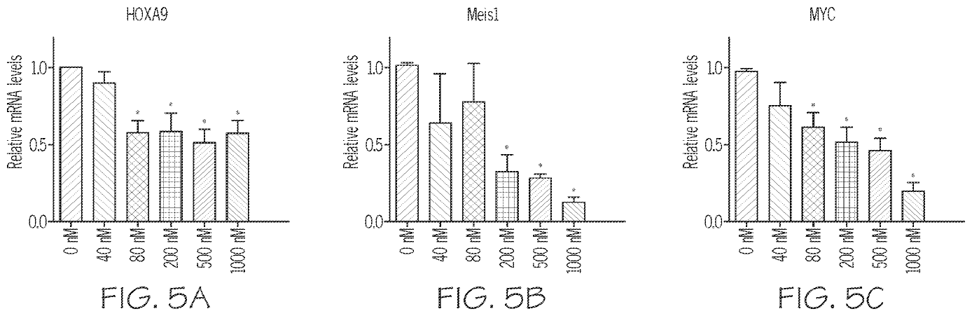

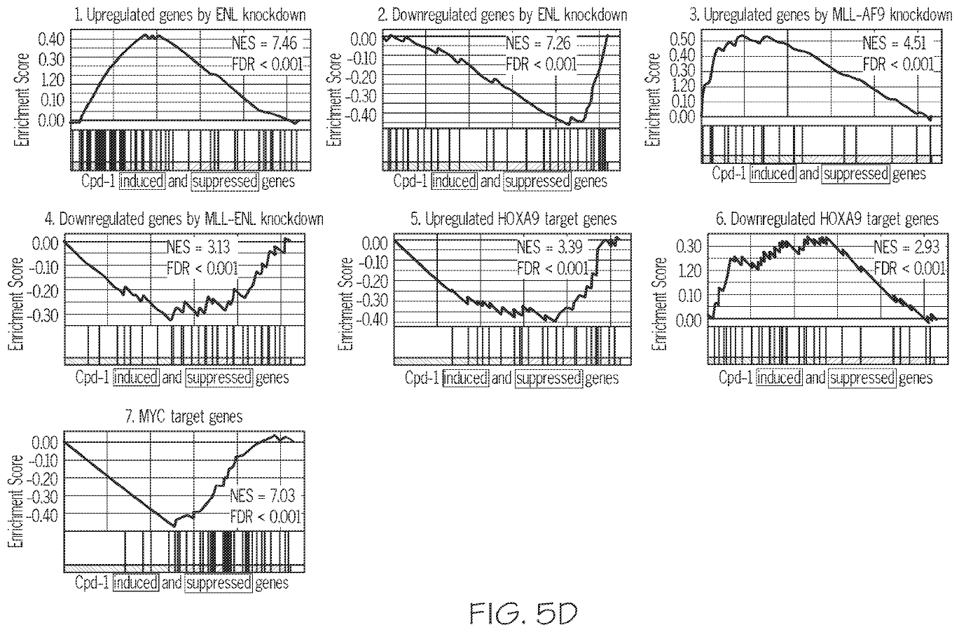

Previous studies show ENL is required for expression of MLL1-target genes in MLL1-r leukemia. How compound 1-mediated ENL degradation changes expression of HoxA9, Meis1 and Myc, three characteristic genes in MLL1-r leukemia, was examined. Molm-13 cells were treated with compound 1 for 4 days, after which the RNAs were extracted and analyzed. As shown in FIGS. 5A-5C, compound 1 was able to significantly inhibit expression of these three genes in a generally dose-dependent manner.

Gene profiling was performed to find how compound 1 affects global gene transcriptome. mRNAs from the control and compound 1 (500 nM) treated Molm-13 cells were extracted, purified and sequenced. Bioinformatic analysis was performed to find differentially expressed genes between the treated and control cells, which were used for gene set enrichment analysis (GSEA). The results are shown in FIG. 5D. Compound 1 caused significant upregulation of a gene set that was upregulated upon knockout of ENL, with normalized enrichment score (NES) of 7.46 and false discovery rate (FDR) of <0.001 (Panel 1 of FIG. 5D). It also led to significant downregulation of a gene set that was downregulated upon ENL knockout with NES of −4.26 and FDR of <0.001 (Panel 2 of FIG. 5D). These clearly indicate that treatment with compound 1 recapitulated ENL knockout with similar patterns of gene expression changes. In addition, compound 1 significantly up- and down-regulated the gene sets that were up- and down-regulated by knockdown of MLL-AF9 or -ENL (Panels 3 and 4 of FIG. 5D), showing the compound treatment mimicked knockdown of these fusion oncogenes. Moreover, compound 1 counteracted two critical transcription factors HoxA9 and Myc in MLL1-r leukemia: treatment with compound 1 reversed expression patterns of HoxA9-regulated genes (Panels 5 and 6 of FIG. 5D). It also significantly downregulated transcription of Myc-target genes (Panel 7 of FIG. 5D). These results are consistent with ENL's critical roles in MLL1-r leukemia and show compound 1 acted on-target.

### Example 1.6. Cpd-1 Inhibits Cell Proliferation, Induces Differentiation and Apoptosis of MLL1-r Leukemia Cells

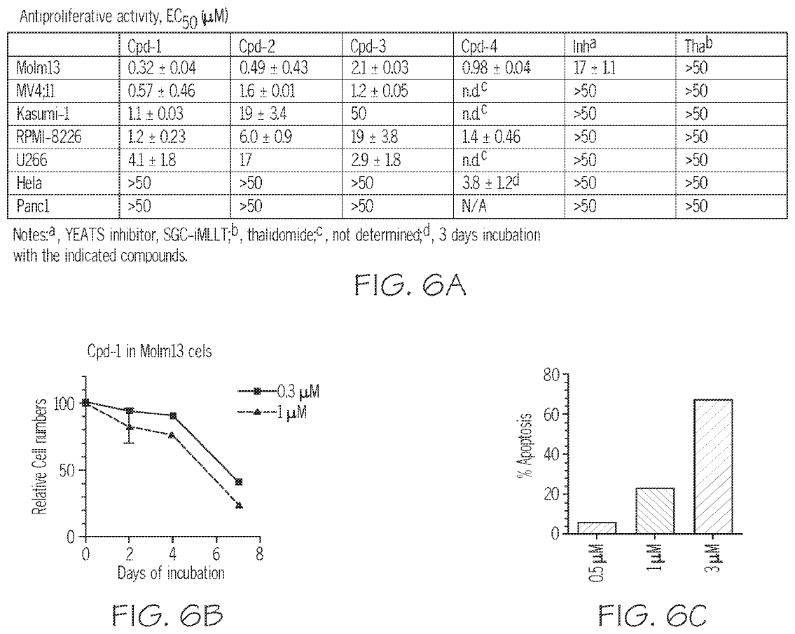

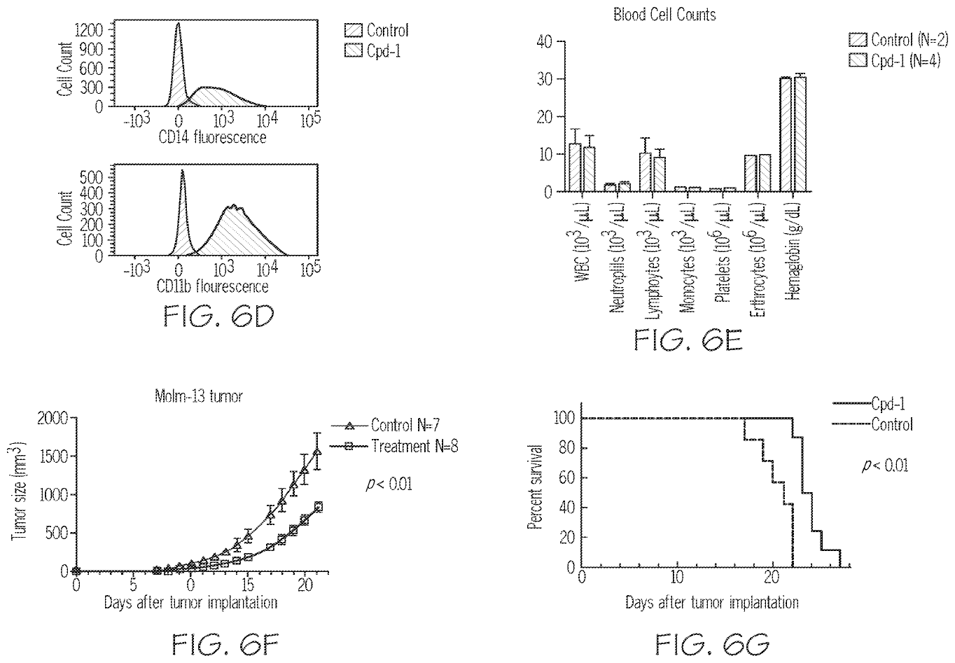

Compound 1 exhibited potent activity against proliferation of MLL1-r leukemia cells Molm-13 and MV4;11 with EC₅₀ values of 320 and 570 nM (FIG. 6A), while the parent inhibitor SGC-iMLLT and thalidomide were inactive (EC₅₀>50 μM) except that SGC-iMLLT had weak activity against Molm-13 cells. Compound 1 also showed strong activity (EC₅₀=1.1-4.1 μM) against AML Kasumi-1 and myeloma RPMI-8226 and U266 cells, in which Myc is critical. However, solid tumor cells Hela (cervical) and Panc1 (pancreatic) are insensitive to compound 1 with EC₅₀ of >50 μM. The selective antitumor activities of 1 is consistent with the critical functions of ENL (or SEC) in MLL1-r leukemia and Myc-driven cancers. Less active compounds 2 and 3 possess a similar antitumor profile with generally reduced potencies (FIG. 6A). Compound 4, which failed to degrade ENL, exhibited strong, but non-selective activities (EC₅₀=1.0-3.8 μM) for these blood and solid tumor cells, presumably due to off-target effects.

As with many compounds targeting gene expression (e.g., epigenetic inhibitors of DOT1L or LSD1), compound 1 exhibited a slow action against cell proliferation. It did not inhibit cell proliferation during the first 4 days, but showed potent activity upon a longer treatment (FIG. 6B). In contrast, compound 4 is a cytotoxic agent, killing cancerous cells non-selectively within 3 days. These results support that the antiproliferative activity of compound 1 is on-target: it degrades ENL and causes suppression of aberrant gene expression and eventually inhibition of cell proliferation at a later stage.

Treatment of Molm-13 cells with compound 1 for 7 days at 1 and 3 μM caused significant apoptosis of 22.7% and 67.6%, respectively (FIG. 6C). No significant apoptosis (<5%) was observed at 0.5 μM or with a shorter incubation (e.g., for 4 days). Compound 1 (3 μM for 5 days) also induced cell differentiation, with significantly more cells having high levels of CD14 and CD11b, two cell surface proteins characteristic to differentiated macrophages or monocytes (FIG. 6D).

### Example 1.7. Cpd-1 Inhibits Tumor Growth in a Mouse Model of MLL1-r Leukemia

In vivo antitumor activity of compound 1 was evaluated in a commonly used mouse model of Molm-13 leukemia. First, in vivo toxicity was assessed in C57BL/6 mice. Treatment with compound 1 (30 mg/kg/day for 13 days) did not cause significant weight losses as well as any visible signs of toxicity. A blood test on day-14 showed that there were no significant differences in blood cell counts between mice in the treatment and control groups (FIG. 6E). These results suggest compound 1 at this dosage did not inhibit normal hematopoiesis or cause other overt toxicities to mice. Next, 10⁶ Molm-13 cells were injected subcutaneously into NOD-SCID mice, which developed palpable tumors in ˜1 week and grew rapidly. As shown in FIGS. 6F-6G, treatment with compound 1 (30 mg/kg/day for Day 3-15) significantly inhibited tumor growth in mice with prolonged animal survivals (p<0.01). Similarly, it did not cause significant weight losses in these animals.

### Example 1.8. Cpd-1 Also Degraded Mutant ENL and Suppressed its Mediated Gene Transcription

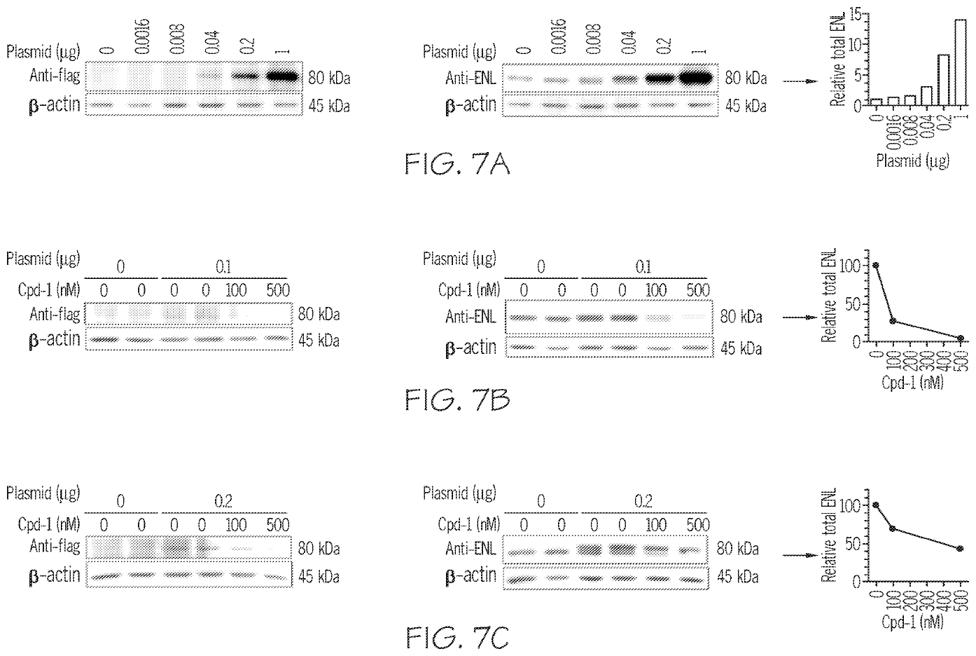

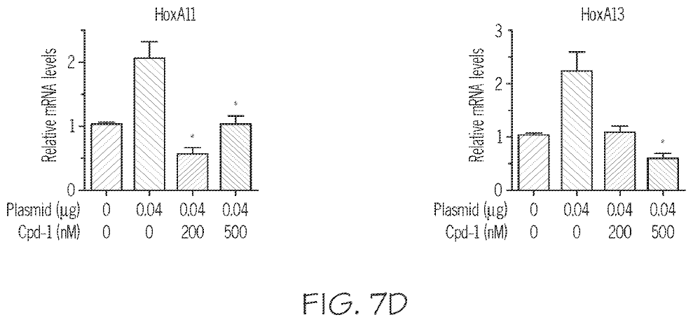

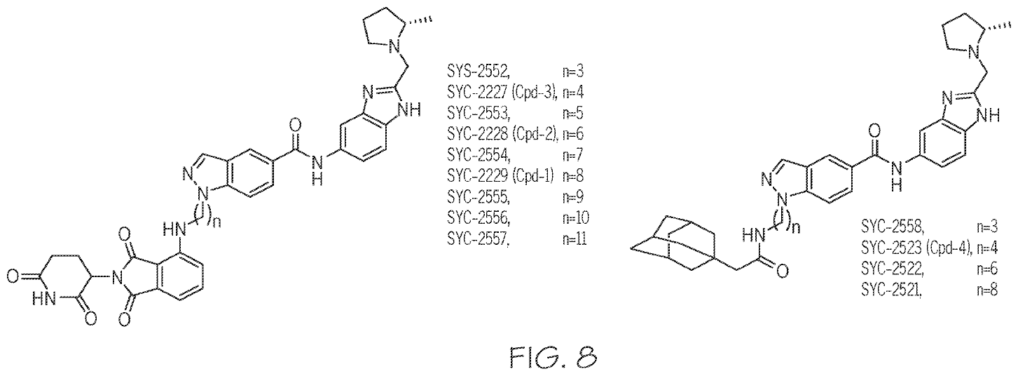

Ability of compound 1 to degrade mutant ENL, which has been implicated to cause Wilms tumor, was evaluated. Frequent clinical ENL mutants contain a short in-frame insertion or deletion in the YEATS domain, but they retain similar binding affinities to the parent inhibitor SGC-iMLLT. A pcDNA3.1(+)-N-DYK plasmid containing a mutant ENL (mENL) with a short insertion of -NHL- between L117 and R118 was transfected into 5×10⁵ HEK293T cells. Upon incubation for 24 h, expression of the FLAG-tagged mENL can be dose-dependently detected with as low as 0.04 g of the plasmid (FIG. 7A). Using a FLAG antibody appeared to be less quantitative because of a higher background staining for the control samples (FIGS. 7A-7C, left panels). While both endogenous, wild-type (WT) ENL and mENL (which cannot be separated by SDS-PAGE) can be detected by an ENL antibody recognizing the peptide residues surrounding A343, the blots had a clean background for quantification (right panels).

With 0.1 μg of the plasmid, compound 1 can efficiently degrade both WT and mutant ENL proteins with Dₘₐₓ of ˜95% at ˜500 nM upon 24 h incubation (FIG. 7B). With 0.2 μg of the plasmid, compound 1 (500 nM) can still degrade WT and mutant ENLs, but with a reduced efficiency (FIG. 7C). It was ineffective with 1 g of the plasmid (not shown), presumably because the rate of mENL synthesis in cells was higher than that of 1-mediated degradation. Since mENL expression in Wilms tumor is comparable to that of WT ENL in normal tissues and 0.2 μg of the plasmid produced considerably more mENL than endogenous ENL (FIG. 7A, right), it is expected that compound 1 can efficiently degrade and even deplete mENL in Wilms tumor.

Expression of mENL has been found to upregulate certain Hox genes, such as HoxA11 and HoxA13, in Wilms tumor and drive oncogenesis. Next, Applicant investigated how mENL degradation affect expression of HoxA11 and HoxA13 in this cell model. Upon transfection with 0.04 g of the plasmid followed by 24 h incubation, expression of HoxA11 and HoxA13 was found to be significantly upregulated (FIG. 7D). Treatment with compound 1 during the incubation significantly inhibited overexpression of HoxA11 and HoxA13 (FIG. 7D), showing degradation of mENL could downregulate aberrant gene expression in Wilms tumor.

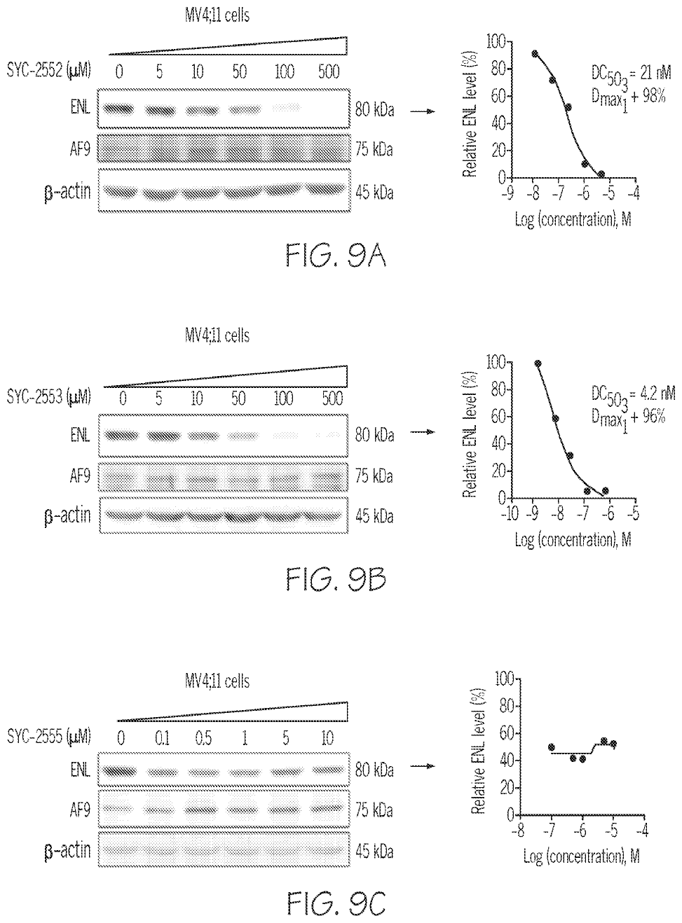

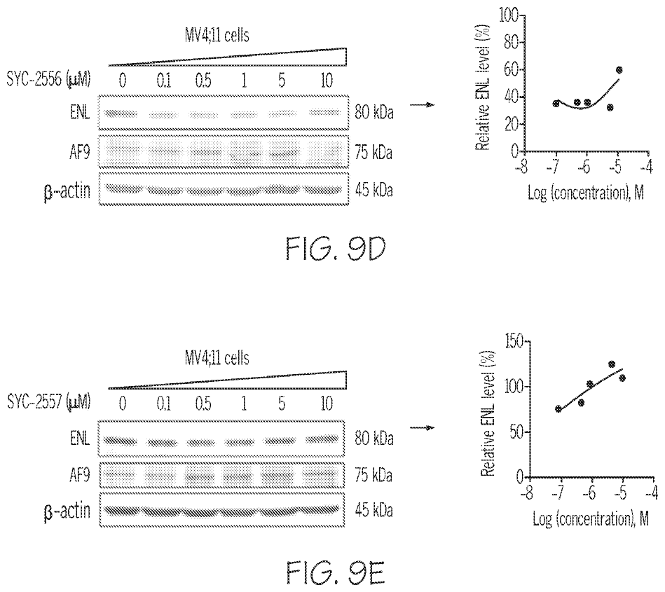

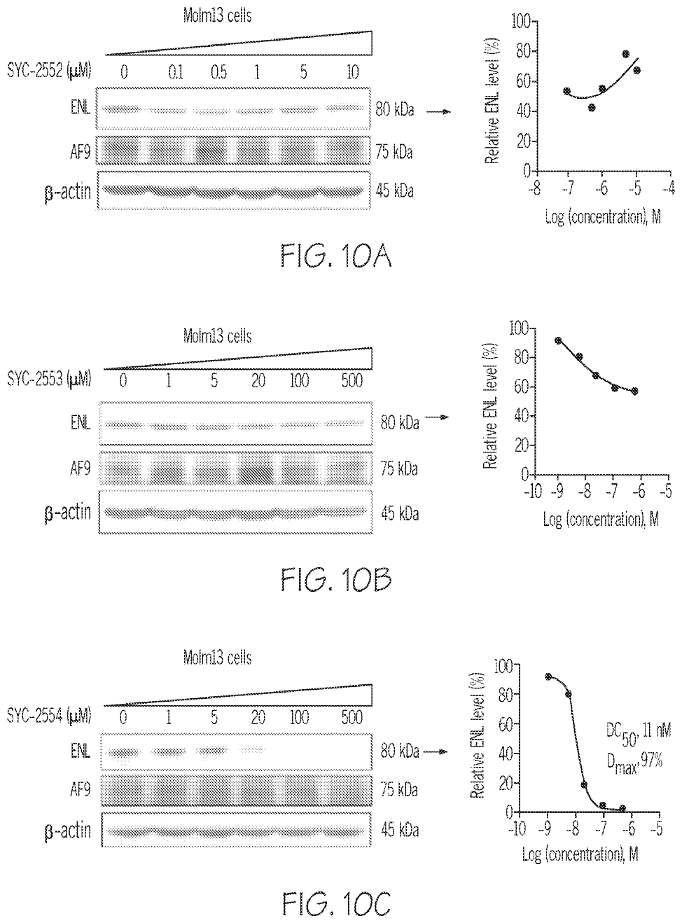

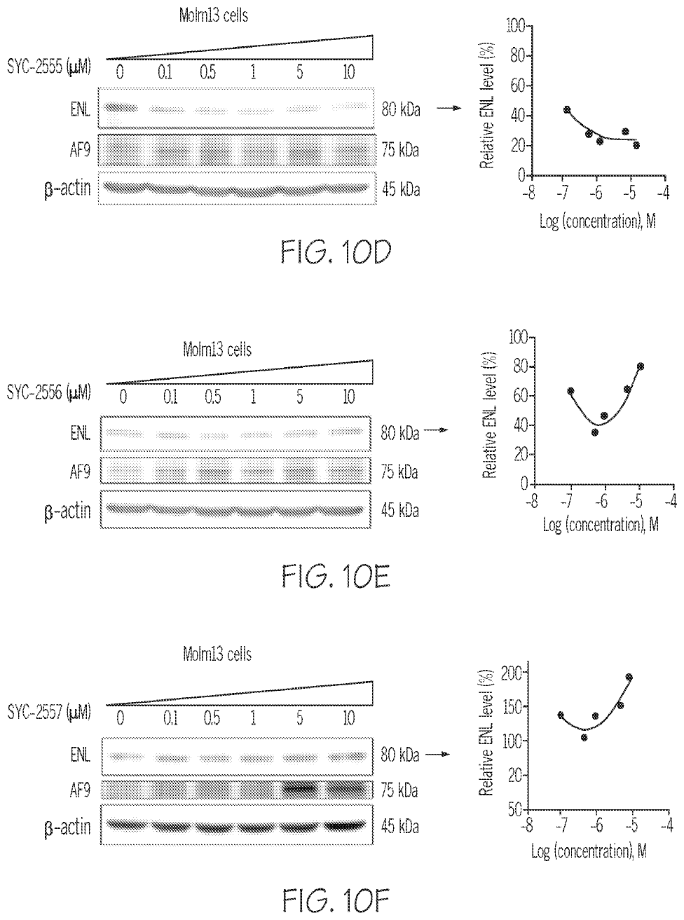

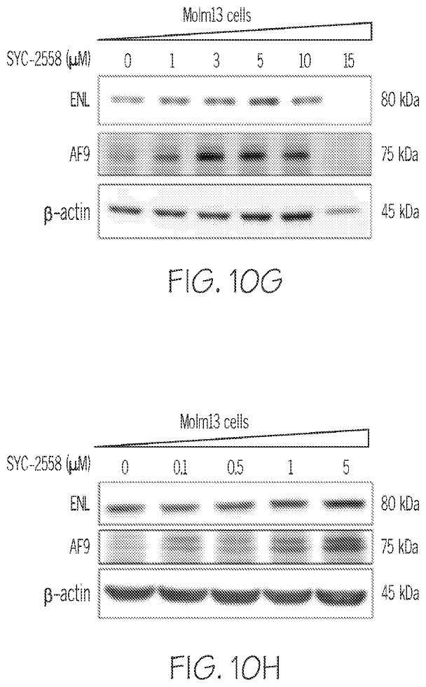

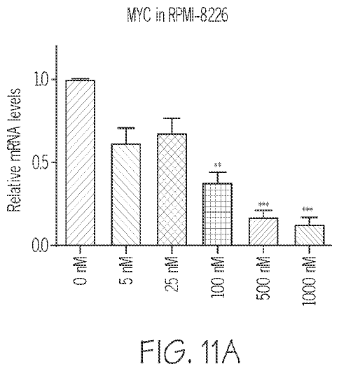

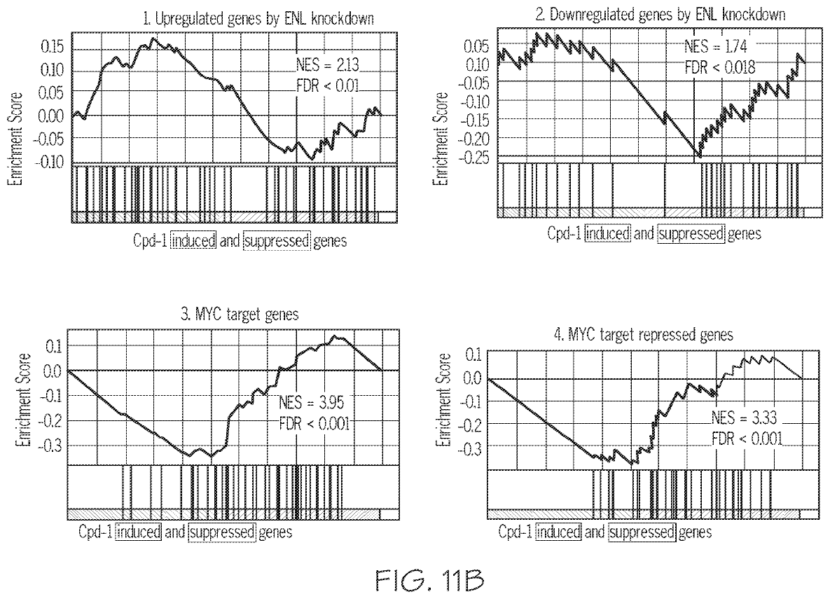

Applicant tested the ENL degradation activities of additional molecules shown in FIG. 8. FIGS. 9A-9E illustrate the ENL or AF9 degradation activities of SYC-2552, SYC-2553, SYC-2555, SYC-2556, and SYC-2557 in MV4;11 cells. Compounds SYC-2552 and -2553 can efficiently degrade ENL with DC₅₀ values of 21 and 4.2 nM, respectively. However, SYC-2555, -2556 and -2557 did not reduce ENL. Similar to SYC-2229 (Cpd-1), all these compounds do not affect AF9. FIGS. 10A-10H illustrate the ENL or AF9 degradation activities of SYC-2552, SYC-2553, SYC-2554, SYC-2555, SYC-2556, SYC-2557, and SYC-2558 in Molm13 cells, Additionally, FIGS. 11A-B demonstrate that SYC-2229 (Compound 1) inhibited malignant gene expression in multiple myeloma RPMI-8226 cells. SYC-2229 dose-dependently decreased expression of MYC, which is critical to multiple myeloma. Moreover, RNA-seq results show treatment of RPMI-8226 cells with SYC-2229 exhibited similar gene expression changes to ENL knockdown (FIG. 11B, upper panel). The compound treatment also significantly down-regulated expression of MYC target gene set (FIG. 11B, lower panel). Table 1 summarizes the anti-proliferative activities of the aforementioned compounds against different cell lines.

TABLE 1
Anti-proliferative activities of compounds (EC50, μM) against
MLL-rearranged leukemia Molm-13 and MV4; 11 cells, multiple
myeloma RPMI-8226 cells, and cervical cancer Hela cell.

| Compound | Molm13 | MV4; 11 | RPMI-8226 | Hela |
|---|---|---|---|---|
| SYC-2552 | 2.5 ± 1.1 | 50 | 50 ^{d} | n.d.^{a} |
| SYC-2227 | 2.1 ± 0.03^{b} | 1.2 ± 0.05^{b} | 19 ± 3.8 | >50 |
| SYC-2553 | 3.4 ± 0.4 | 8.3 ± 1.6 | 7.2 ± 0.35 | n.d.^{a} |
| SYC-2228 | 0.49 ± 0.43^{b} | 1.6 ± 0.01^{b} | 6.0 ± 0.91 | >50^{b} |
| SYC-2554 | 4.4 ± 0.5 | 3.7 ± 1.6 | 6.4 ± 0.65 | n.d.^{a} |
| SYC-2229 | 0.32 ± 0.04^{b} | 0.57 ± 0.46^{b} | 1.2 ± 0.23 | >50^{b} |
| SYC-2555 | 4.5 ± 0.6 | 8.3 ± 0.8 | 7.9 ± 0.07 | n.d.^{a} |
| SYC-2556 | 1.1 ± 0.3 | 8.4 ± 1.2 | 7.5 ± 0.19 | n.d.^{a} |
| SYC-2557 | 0.82 ± 0.2 | 0.94 ± 0.06 | 5.5 ± 0.11 ^{d} | n.d.^{a} |
| SYC-2558 | n.d.^{a} | n.d.^{a} | n.d.^{a} | 2.5 ± 1.0^{c} |
| SYC-2523 | 0.98 ± 0.04 | n.d.^{a} | 1.4 ± 0.5^{c} | 3.8 ± 1.2^{b, c} |
| SYC-2522 | 1.0 ± 0.05 | n.d.^{a} | 0.68 ± 0.2 | 3.2 ± 0.7^{c} |
| SYC-2521 | 1.0 ± 0.2 | n.d.^{a} | 2.0 ± 0.3 | 2.5 ± 1.4^{c} |
| SGC-iMLLT | 17 ± 1.1^{b} | >50^{b} | >50^{b} | >50^{b} |
| Thalidomide | >50^{b} | >50^{b} | >50^{b} | >50^{b} |

Note:
^{a}not determined;
^{b}reported in Li, et al. J Hematol Oncol, 2022.;
^{c}3 days incubation with the indicated compounds,
^{d} 6 days incubation with the indicated compounds

### Example 1.9. Summary

In this Example, various compounds were found to be a highly efficient, ENL-specific PROTAC molecules, able to degrade ENL with DC₅₀ values as low as 37 nM and deplete it at ˜500 nM (Dₘₐₓ˜95%) in a variety of blood and solid tissue cells. AF9 (as well as other proteins in SEC) was not significantly reduced (e.g., FIGS. 4A-4B). ChIP experiments further indicate that the compounds only reduced the ENL levels in several MLL1 target gene promoters (e.g., FIG. 4C). Compound-mediated ENL depletion significantly suppressed aberrant gene signatures in MLL1-r leukemia, including reduced expression of several characteristic genes (e.g., HoxA9 and Myc) (e.g., FIGS. 5A-5D). Compound-mediated global gene expression changes caused inhibited cell proliferation (with EC₅₀s as low as 320 nM) and cell differentiation and apoptosis. The compounds also showed significant antitumor activity in a mouse model of MLL1-r leukemia without inhibition of normal hematopoiesis and other overt toxicities (FIGS. 6E-6G). These results are consistent with ENL's essential roles in MLL1-r leukemia and other cancers (e.g., Myc-driven cancers) and support AF9 is indeed dispensable in these contexts. Thus, the compounds tested are the first potent chemical probe for cellular and in vivo studies of ENL's functions in health and diseases. The compounds also represent a pharmacological lead for drug development for these cancers.

In summary, Applicant developed potent PROTAC molecules for selective ENL degradation. The compounds strongly inhibited malignant gene expression and cell proliferation of MLL1-r leukemia and Myc-driven cancers.

Without further elaboration, it is believed that one skilled in the art can, using the description herein, utilize the present disclosure to its fullest extent. The embodiments described herein are to be construed as illustrative and not as constraining the remainder of the disclosure in any way whatsoever. While the embodiments have been shown and described, many variations and modifications thereof can be made by one skilled in the art without departing from the spirit and teachings of the invention. Accordingly, the scope of protection is not limited by the description set out above, but is only limited by the claims, including all equivalents of the subject matter of the claims. The disclosures of all patents, patent applications and publications cited herein are hereby incorporated herein by reference, to the extent that they provide procedural or other details consistent with and supplementary to those set forth herein.

What is claimed is:

1. A compound comprising:
a molecule capable of binding to an ENL protein; and
a ligand of an E3 ubiquitin ligase, wherein the molecule and the ligand are coupled to one another by a linker, a chemical bond, or combinations thereof.

2. The compound of claim 1, wherein the molecule comprises a structure of: 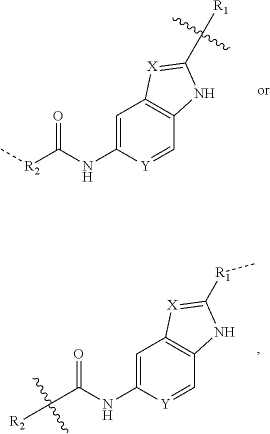
wherein [symbol] represents a chemical bond or linker that couples the molecule to the linker;
wherein R₁ comprises a functional group selected from the group consisting of 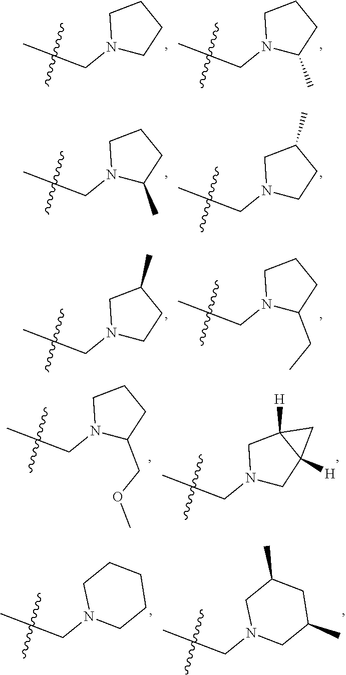
derivatives thereof, or combinations thereof;
wherein R₂ is selected from the group consisting of 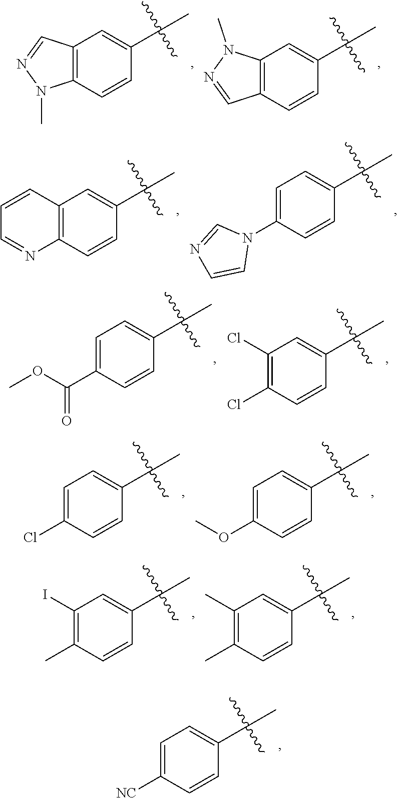
derivatives thereof, or combinations thereof; and
wherein each of X and Y is independently selected from the group consisting of N or CH.

3. The compound of claim 2, wherein [symbol] represents a chemical bond between the molecule and the ligand.

4. The compound of claim 2, wherein [symbol] represents a linker.

5. The compound of claim 4, wherein the linker comprises a structure selected from the group consisting of 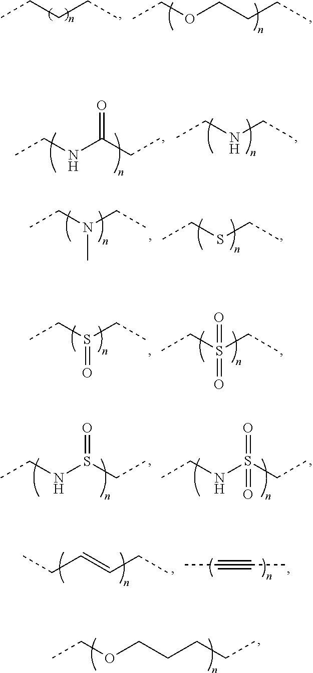
derivatives thereof, or combinations thereof, and
wherein n is an integer of 1 or greater.

6. The compound of claim 5, wherein n is an integer between 1-20.

7. The compound of claim 1, wherein the ligand is selected from the group consisting of a von Hippel-Lindau disease tumor suppressor protein (VHL) ligand, a cereblon (CRBN) ligand, the mouse double minute 2 homologue (MDM2), inhibitor of apoptosis (IAP) ligand, or combinations thereof.

8. The compound of claim 1, wherein the ligand comprises a VHL ligand.

9. The compound of claim 8, wherein the VHL ligand is selected from the group consisting of 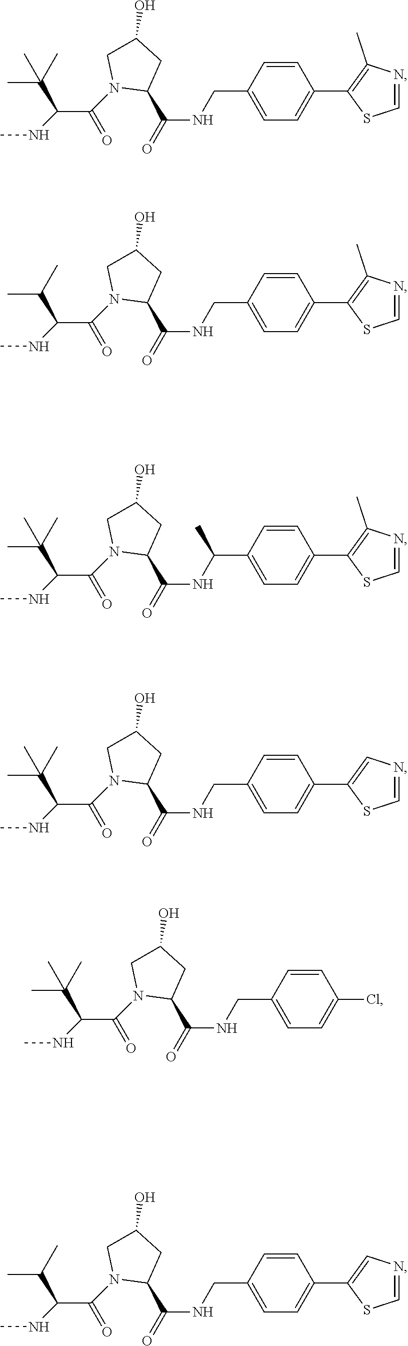 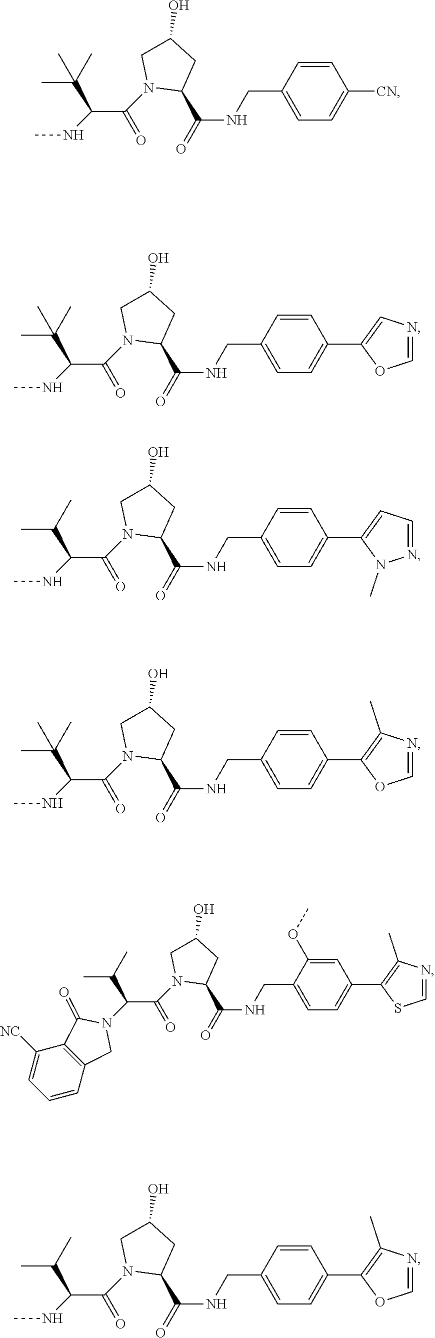
derivatives thereof, or combinations thereof, and
wherein [symbol] represents a chemical bond or a linker.

10. The compound of claim 1, wherein the ligand comprises a CRBN ligand.

11. The compound of claim 10, wherein the CRBN ligand is selected from the group consisting of 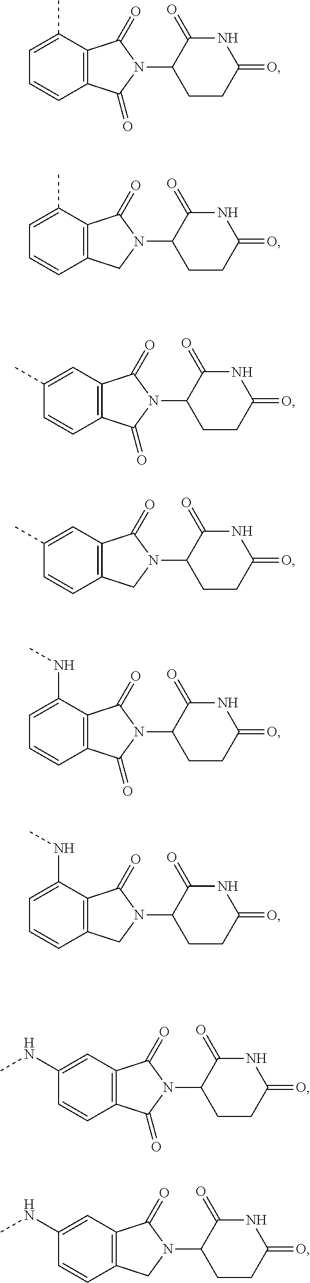 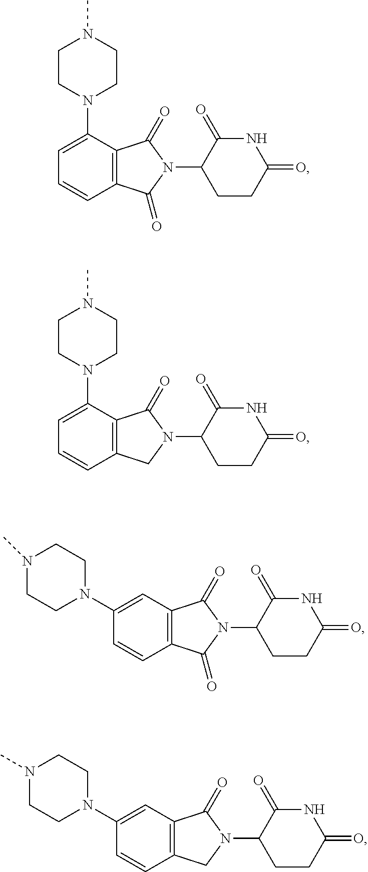
derivatives thereof, or combinations thereof, and
wherein [symbol] represents a chemical bond or a linker.

12. The compound of claim 1, wherein the compound comprises the following structure: 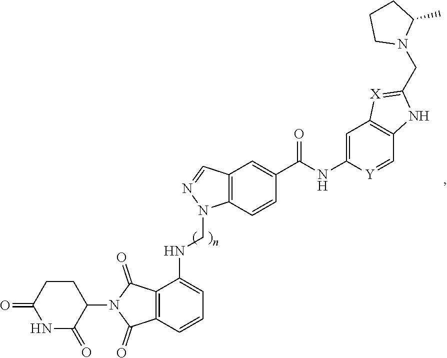
wherein n is an integer of 0 or greater, and
wherein each of X and Y is independently selected from the group consisting of N or CH.

13. The compound of claim 12, wherein n is 3, 4, 5, 6, 7, 8, 9, 10 or 11.

14. The compound of claim 12, wherein n is 4, 6, or 8.

15. The compound of claim 12, wherein n is 8.

16. The compound of claim 1, wherein the compound is suitable for use in treating or preventing a condition in a subject, wherein the condition is associated with an ENL protein abnormality or facilitated by an ENL protein.

17. The compound of claim 1, wherein the ENL protein comprises SEQ ID NO: 1, a sequence that shows at least 65% similarity to SEQ ID NO: 1, a derivative thereof, a homologue thereof, an analog thereof, or combinations thereof.

18. A method of treating or preventing a condition in a subject, said method comprising:
administering to the subject a compound, wherein the compound comprises:
a molecule capable of binding to an ENL protein; and
a ligand of an E3 ubiquitin ligase,
wherein the molecule and the ligand are coupled to one another by a linker, a chemical bond, or combinations thereof, and
wherein the condition is associated with an ENL protein abnormality or facilitated by an ENL protein.

19. The method of claim 18, wherein the ENL protein comprises SEQ ID NO: 1, a sequence that shows at least 65% similarity to SEQ ID NO: 1, a derivative thereof, a homologue thereof, an analogue thereof, or combinations thereof.

20. The method of claim 18, wherein the condition is associated with an ENL protein abnormality.

21. The method of claim 20, wherein the ENL protein abnormality is characterized by overexpression of the ENL protein, under-expression of the ENL protein, mutation of the ENL protein, or combinations thereof.

22. The method of claim 18, wherein the condition is facilitated by an ENL protein.

23. The method of claim 22, wherein the ENL protein facilitates, propagates, or causes the condition.

24. The method of claim 18, wherein the condition is a cancer.

25. The method of claim 24, wherein the cancer is selected from the group consisting of a cancer facilitated by an ENL protein, a cancer associated with an ENL protein abnormality, leukemia, acute lymphocytic leukemia (ALL), myeloid leukemia (AML), mixed lineage leukemia 1 (MLL1), MLL1-rearranged (MLL1-r) ALL, Wilms tumor, kidney cancer, or combinations thereof.

26. The method of claim 24, wherein the cancer comprises mixed lineage leukemia 1 (MLL1).

27. The method of claim 18, wherein the method is used to treat the condition.

28. The method of claim 18, wherein the method is used to prevent the condition.

29. The method of claim 18, wherein the subject is a human being.

30. The method of claim 18, wherein the subject is suffering from the condition.

31. The method of claim 18, wherein the subject is vulnerable to the condition.

32. The method of claim 18, further comprising a step of instructing the subject to administer the compound in order to treat or prevent the condition in the subject.

33. The method of claim 18, wherein the administering occurs by a method selected from the group consisting of intravenous administration, intramuscular administration, intradermal administration, intraperitoneal administration, subcutaneous administration, spray-based administration, aerosol-based administration, in ovo administration, oral administration, intraocular administration, intratracheal administration, intranasal administration, inhalational administration, local administration, and combinations thereof.

34. The method of claim 18, wherein the administering occurs by oral administration.

35. The method of claim 18, wherein the compound comprises the following structure: 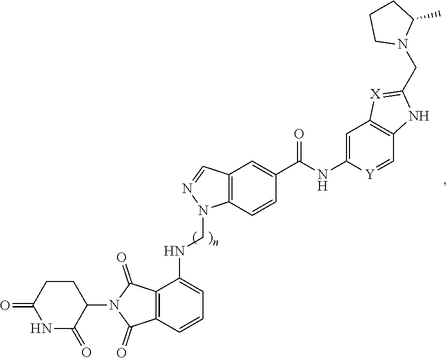
wherein n is an integer of 0 or greater, and
wherein each of X and Y is independently selected from the group consisting of N or CH.

36. A method of evaluating cellular activity, said method comprising:
exposing a cell to a compound, wherein the compound comprises:
a molecule capable of binding to an ENL protein; and
a ligand of an E3 ubiquitin ligase,
wherein the molecule and the ligand are coupled to one another by a linker, a chemical bond, or combinations thereof.

37. The method of claim 36, wherein the exposing occurs in vitro, and wherein the method is utilized to evaluate the cellular activity in vitro.

38. The method of claim 36, wherein the exposing occurs in vivo in a subject, and wherein the method is utilized to evaluate the cellular activity in vivo.

39. The method of claim 36, wherein the cell comprises a cancer cell, and wherein the cellular activity comprises carcinogenesis.

40. The method of claim 39, wherein the method is utilized to evaluate the ability of the compound to interfere with the carcinogenesis.

41. The method of claim 38, wherein the compound comprises the following structure: 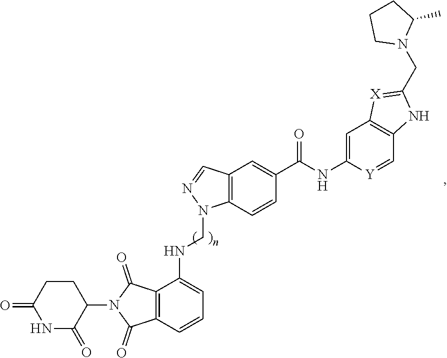
wherein n is an integer of 0 or greater, and
wherein each of X and Y is independently selected from the group consisting of N or CH.


SEQUENCE LISTING (1 sequence)

SEQ ID NO: 1 - protein, 559 aa, Homo sapiens
MDNQCTVQVRLELGHRAQLRKKPTTEGFTHDWMVFVRGPEQCDIQHFVEKVVFWLHDSFPKPRRVCKEPPYKVEESGYAGFIMPIEVHFKNKEEPRKVCFTYDLFLNLEGNPPVNHLRCEKLTFNNPTTEFRYKLLRAGGVMVMPEGADTVSRPSPDYPMLPTIPLSAFSDPKKTKPSHGSKDANKESSKTSKPHKVTKEHRERPRKDSESKSSSKELEREQAKSSKDTSRKLGEGRLPKEEKAPPPKAAFKEPKMALKETKLESTSPKGGPPPPPPPPPRASSKRPATADSPKPSAKKQKKSSSKGSRSAPGTSPRTSSSSSFSDKKPAKDKSSTRGEKVKAESEPREAKKALEVEESNSEDEASFKSESAQSSPSNSSSSSDSSSDSDFEPSQNHSQGPLRSMVEDLQSEESDEDDSSSGEEAAGKTNPGRDSRLSFSDSESDNSADSSLPSREPPPPQKPPPPNSKVSGRRSPESCSKPEKILKKGTYDKAYTDELVELHRRLMALRERNVLQQIVNLIEETGHFNVTNTTFDFDLFSLDETTVRKLQSCLEAVAT